SEQUENCE LISTING (15 sequences)

SEQ ID NO: 1 - protein, 12 aa, Homo sapiens
  DISULFID 1..12: note = Intrachain disulfide bond
  SITE 1: note = C residue linked via amide linkage to dipeptide AG
CKNFFWKTFTSC

SEQ ID NO: 2 - protein, 16 aa, Homo sapiens
  SITE 16: note = G residue at position 16 linked via amide linkage to C(1) of SEQ ID NO. 3
SANSNPAMAPRERKAG

SEQ ID NO: 3 - protein, 12 aa, Homo sapiens
  DISULFID 1..12: note = intrachain disulfide bond
  SITE 1: note = Residue C(1) linked via amide linkage to G(16) of SEQ ID NO. 2
CKNFFWKTFTSC

SEQ ID NO: 4 - protein, 6 aa, Homo sapiens
  DISULFID 1..6: note = Intrachain disulfide bond
  SITE 3: note = D-Tryptophan
  SITE 1: note = Bonded via amide linkage to D-phenylalanine
  SITE 6: note = Bonded via amide linkage to Threonine-OH
CYWKTC

SEQ ID NO: 5 - protein, 6 aa, Homo sapiens
  DISULFID 1..6: note = Intrachain disulfide bond
  SITE 3: note = D-Tryptophan
  SITE 1: note = Bonded via amide linkage to D-Phenylalanine
  SITE 6: note = Bonded via amide linkage to Threonine
CYWKTC

SEQ ID NO: 6 - protein, 6 aa, Homo sapiens
  DISULFID 1..6: note = Intrachain disulfide bond
  SITE 3: note = D-Tryptophan
  SITE 2: note = 1-Naphthylalanine
  SITE 1: note = Bonded via amide linkage to D-Phenylalanine
  SITE 6: note = Bonded via amide linkage to Threonine-OH
CXWKTC

SEQ ID NO: 7 - protein, 6 aa, Homo sapiens
  DISULFID 1..6: note = Intrachain disulfide bond
  SITE 3: note = D-Tryptophan
  SITE 1: note = Bonded via amide linkage to D-Phenylalanine
  SITE 6: note = Bonded via amide linkage to Threonine-OH
  SITE 2: note = Benzothiopene (BzThi)
CXWKTC

SEQ ID NO: 8 - protein, 6 aa, Homo sapiens
  DISULFID 1..6: note = Intrachain disulfide bond
  SITE 3: note = D-Tryptophan
  SITE 1: note = Bonded to beta-D-Naphthylalanine
  SITE 6: note = Bonded via amide linkage to Threonine-NH2
CYWKVC

SEQ ID NO: 9 - protein, 6 aa, Homo sapiens
  DISULFID 1..6: note = Intrachain disulfide bond
  SITE 3: note = D-Tryptophan
  SITE 1: note = Bonded via amide linkage to D-Phenylalanine
  SITE 6: note = Bonded via amide linkage to Threonine-NH2
CYWKVC

SEQ ID NO: 10 - protein, 6 aa, Homo sapiens
  DISULFID 1..6: note = Intrachain disulfide bond
  SITE 2: note = (3-iodo)-Tyrosine
  SITE 3: note = D-Tryptophan
  SITE 1: note = Bonded via amide linkage to (4-amino-3-iodo)-D-Phenylalanine
  SITE 6: note = Bonded via amide linkage to Threonine-NH2
CYWKVC

SEQ ID NO: 11 - protein, 6 aa, Homo sapiens
  DISULFID 1..6: note = Intrachain disulfide bond
  SITE 1: note = Bonded via amide linkage to (4-amino-3-iodo)-D-Phenylalanine
  SITE 2: note = (3-Cl)-Tyrosine
  SITE 3: note = D-Tryptophan
  SITE 6: note = Bonded via amide linkage to Threonine-NH2
CYWKVC

SEQ ID NO: 12 - protein, 6 aa, Homo sapiens
  DISULFID 1..6: note = Intrachain disulfide bond
  SITE 1: note = Bonded via amide linkage to (4-amino-3-chloro)-D-Phenylalanine
  SITE 2: note = (3-iodo)-Tyrosine
  SITE 3: note = D-Tryptophan
  SITE 6: note = Bonded via amide linkage to Threonine-NH2
CYWKVC

SEQ ID NO: 13 - protein, 6 aa, Homo sapiens
  DISULFID 1..6: note = Intrachain disulfide bond
  SITE 1: note = Bonded via amide linkage to (4-amino-3-chloro)-D-Phenylalanine
  SITE 2: note = (3-chloro)-Tyrosine
  SITE 3: note = D-Tryptophan
  SITE 6: note = Bonded via amide linkage to Threonine-NH2
CYWKVC

SEQ ID NO: 14 - protein, 6 aa, Homo sapiens
  DISULFID 1..6: note = Intrachain disulfide bond
  SITE 1: note = Bonded via amide linkage to (4-amino-3-fluoro)-D-Phenylalanine
  SITE 2: note = (3-fluoro)-Tyrosine
  SITE 3: note = D-Tryptophan
  SITE 6: note = Bonded via amide linkage to Threonine-NH2
CYWKVC

SEQ ID NO: 15 - protein, 6 aa, Homo sapiens
  DISULFID 1..6: note = Intrachain disulfide bond
  SITE 1: note = Bonded via amide linkage to (4-amino-3-bromo)-D-Phenylalanine
  SITE 2: note = (3-bromo)-Tyrosine
  SITE 3: note = D-Tryptophan
  SITE 6: note = Bonded via amide linkage to Threonine-NH2
CYWKVC

(19) United States
(12) Patent Application Publication
(10) Pub. No.: US 2026/0053958 A1
(43) Pub. Date: Feb. 26, 2026

(54) HALOGENATED SOMATOSTATIN ANALOGS WITH MULTIPLE SOMATOSTATIN RECEPTOR SUBTYPE SELECTIVITY

(71) Applicant: GRAFTON THERAPEUTICS SÀRL, Saint-Prex (CH)

(72) Inventors: Alan HARRIS, New York, NY (US); Adrian DALY, Gijón (ES)

(21) Appl. No.: 19/106,024

(22) PCT Filed: Aug. 30, 2023
(86) PCT No.: PCT/EP2023/073801

§ 371 Date: Feb. 24, 2025

Related U.S. Application Data
(60) Provisional application No. 63/373,945, filed on Aug. 30, 2022.

(51) Int. Cl. A61K 51/08; A61P 35/00

(52) U.S. Cl. CPC A61K 51/083; A61K 51/088; A61P 35/00; A61K 2121/00

(57) ABSTRACT

The present invention relates to halogenated somatostatin analogs with selectivity for multiple somatostatin receptor subtypes and pharmaceutical compositions comprising the same. Moreover, the present invention relates to said halogenated somatostatin analogs and pharmaceutical compositions for use in therapy and/or diagnosis, for example in the treatment and/or diagnosis of diseases (e.g., cancer such as a neuroendocrine cancer) that are characterized by a high expression (e.g., overexpression) of one or more somatostatin receptor subtypes (e.g., somatostatin receptor 2 (SST2) and/or somatostatin receptor 5 (SST5)).

The present invention relates to halogenated somatostatin analogs with multiple somatostatin receptor subtype selectivities and pharmaceutical compositions comprising the same. Moreover, the present invention relates to said halogenated somatostatin analogs and pharmaceutical compositions for use in diagnosis and/or treatment, for example in the diagnosis and/or treatment of diseases that are characterized by a high expression (e.g., overexpression) of one or more somatostatin receptor subtypes (e.g., somatostatin receptor 2 (SST2) and/or somatostatin receptor 5 (SST5)).

### RELATED ART

The somatostatin receptors belong to the family of G-protein coupled receptors. Five somatostatin receptor subtypes (SST1-SST5) are known (Reubi J C. Endocr. Rev. 2003; 24(4):389-427; Schaer J C, et al. Int. J. Cancer. 1997; 70(5):530-7). Somatostatin itself consists of two cyclic disulphide-containing peptide hormones, one with 14 amino acids (SS-14, Ala-Gly-c(Cys-Lys-Asn-Phe-Phe-Trp-Lys-Thr-Phe-Thr-Ser-Cys)) and one with 28 amino acids (SS-28, Ser-Ala-Asn-Ser-Asn-Pro-Ala-Met-Ala-Pro-Arg-Glu-Arg-Lys-Ala-Gly-c(Cys-Lys-Asn-Phe-Phe-Trp-Lys-Thr-Phe-Thr-Ser-Cys). When activated in vivo, for instance upon binding to SS-14 or SS-28, somatostatin receptors (e.g., SST2 and SST5) produce biological effects, for example inhibiting the secretion of hormones such as growth hormone (GH), thyroid stimulating hormone (TSH), adrenocorticotropic hormone (ACTH), prolactin (PRL), gastrin, insulin, and/or glucagon.

All five somatostatin receptor subtypes are expressed or overexpressed in varying frequencies in different normal and diseased tissues. For instance, in neuroendocrine tumors (NETs), SST2 is often the most prominent subtype in differentiated NET. However, although the majority of NETs express SST2, their distribution and density are often variable (Reubi J C, Waser B. Eur. J. Nucl. Med. Mol. Imaging. 2003; 30:781-793). Moreover, different somatostatin receptor subtypes SST1-SST5 showed different expression patterns in various tumors and even within the same tumor type. SST2 and SST5, for example, are expressed at a high density in 70-100% of gastroenteropancreatic neuroendocrine tumors (GEP-NETs) and are often expressed together in growth hormone (GH)-secreting pituitary adenomas (Reubi J C, Waser B. Eur. J. Nucl. Med. Mol. Imaging. 2003; 30:781-793).

Because of somatostatin's ability to inhibit secretion of growth hormone and the overexpression of somatostatin receptors in tumors such as NETs, there has been interest in using somatostatin for the treatment of proliferative disease. However, somatostatin itself is unsuitable for in vivo use, given the short plasma half-life of 1-3 min (Benuck & Marks, Life Sciences. 1976; 19(8):1271-1276). As a consequence, a number of somatostatin analogs more resistant to enzymatic degradation than somatostatin itself have been developed during the last decades by modifications of the natural molecule including the introduction of D-amino acids, chemical modifications not only but in particular of the N terminus of the molecule, variation in chain length, and the like (Esther I. Van Vliet, et al; Somatostatin Analogues and Radionuclides Used in Therapy, Wiley, 2015). Unlike native somatostatin that binds with high affinity to all five SSTs, certain somatostatin analogs octreotide and lanreotide bind with high affinity preferentially to SST2.

Somatostatin analogs can also be used in diagnostic and theranostic settings, such scintigraphy, positron emission tomography (PET) imaging and single photon emission computed tomography (SPECT) imaging and therapeutic settings, such as peptide receptor radionuclide therapy (PRRT or PRRNT). In such contexts, somatostatin analogs are labeled with different radionuclides, most often radiometals. In order to effect said labeling, the somatostatin analogs are typically conjugated with a chelator that is able to form stable complexes with the radiometals. The selection and use of different radionuclides depend on the specific diagnostic and therapeutic application. Upon conjugation to the somatostatin analog, the chelator immobilizes the corresponding radiometal and the diagnostic or theranostic radiolabeled somatostatin analog is formed.

The first commercially available diagnostic somatostatin analog was the radiolabeled somatostatin analog ¹¹¹In-DTPA-octreotide (DTPA: diethylenetriaminepentaacetic acid) (Octreoscan®). Octreoscan® has become the standard imaging procedure for the management of NET patients and it has high affinity only for the SST2 receptor subtype and shows high accuracy in the diagnosis of SST2-positive primary NETs and secondary lesions.

Other prominent somatostatin analogs which have been developed diagnostically are TOC (D-Phe-cyclo(Cys-Tyr-D-Trp-Lys-Thr-Cys)Thr(ol)) and TATE (D-Phe-cyclo(Cys-Tyr-D-Trp-Lys-Thr-Cys)Thr) showing an improved affinity for the SST2 (Reubi J C, et al. Eur J Nucl Med. 2000; 27(3):273-82). Furthermore, modifications of octreotide at position 3 by introducing the unnatural amino acids 1-naphtyl-alanine (1-Nal) led to the analog NOC (D-Phe-cyclo(Cys-1-Nal-D-Trp-Lys-Thr-Cys)Thr(ol)) (Antunes P, et al. Eur J Nucl Med Mol Imaging. 2007; 34(7):982-93), while the introduction of a benzothiophene (BzThi) group in the same position led to the analog BOC (D-Phe-cyclo(Cys-BzThi-D-Trp-Lys-Thr-Cys)Thr(ol)), both with affinity not only for the SST2 but also for SST3 and SST5 (Ginj M, et al. Chemistry and Biology. 2006; 13:1081-1090). Further modification by introduction of Thr in position 8 instead of a Thr(ol) led to NOC-ATE and BOC-ATE (Ginj M, et al. Chemistry and Biology. 2006; 13:1081-1090, Ginj M, et al. Clin Cancer Res. 2005; 11:1136-1145). Somatostatin analogs having affinity for SST2 are also analogs derived from lanreotide (D-β-Nal-cyclo(Cys-Tyr-D-Trp-Lys-Val-Cys)-Thr-NH₂) (Lamberts S W, et al. N Engl J Med. 1996; 334:246-254) or RC-121 (D-Phe-c[Cys-Tyr-D-Trp-Lys-Val-Cys]-Thr-NH₂) (Cai et al. Proc. Natl. Acad. Sci. 1986; 83:1896-1900). Additionally, Moore et al. developed a series of somatostatin analogs with affinity for the SST2 and SST5 including (4-amino-3-iodo)-D-Phe-c[Cys-(3-iodo)-Tyr-D-Trp-Lys-Val-Cys]-Thr-NH₂) (Moore S B, et al. J Med Chem. 2005; 48:6643-6652). However, a chelated version of this compound was found to be unsuitable for clinical use because chelation interfered with SST5 affinity and led to high uptake in the liver and kidney (Mansi R. et al., Molecules, 2020; 25:4155; Mansi R. et al., EJNMMI Research, 2020; 10:90).

Somatostatin analogs are very sensitive to structural modifications. This includes the design of somatostatin-based diagnostic and theranostic radiopharmaceuticals. The effect of introducing one or more structural modifications on the affinity, biological and in vivo properties of a somatostatin analog is not predictable, and each modification produces a unique pharmaceutical.

Despite the successful developments made, it is of prime interest to further develop somatostatin analogs that bind with high affinity to a broad spectrum of SST subtypes (e.g., SST2 and/or SST5) to increase the therapeutic efficacy of currently available analogs.

### SUMMARY OF THE INVENTION

The present invention relates to halogenated somatostatin analogs that exhibit sub- and low-nanomolar binding affinity to somatostatin receptors SST2 and/or SST5. The binding affinities of the inventive compounds to SST2 and SST5 are therefore comparable to that of SS-28, one of the natural ligands of SST2 and SST5. Accordingly, the inventive compounds are expected to exhibit high uptake, and preferably high activity, in tumor cells (e.g., NET cells and their metastases, preferably wherein said tumor cells express or overexpress SST2 and/or SST5). Moreover, the inventive compounds comprise either one or no iodine atoms. Therefore, the inventive compounds are expected to exhibit reduced update in healthy organs and tissue (e.g., kidney and/or liver) in vivo. Additional features and advantages of the present technology will be apparent to one of skill in the art upon reading the Detailed Description, below.

In one aspect, the present disclosure provides a compound of the Formula I, or a pharmaceutically acceptable salt or solvate thereof:

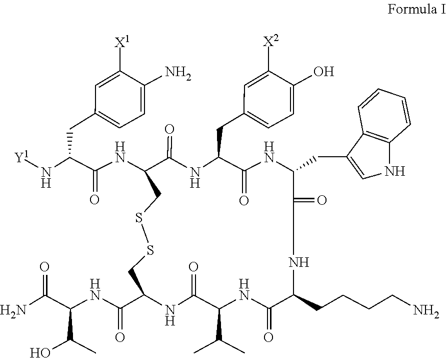

wherein:
X¹ is selected from the group consisting of —F, —Cl, —Br, and —I;
X² is selected from the group consisting of —F, —Cl, —Br, and —I; and
Y¹ is selected from the group consisting of —H, a chelator, L¹-TG, -L¹-chelator, an amino protecting group, and a solid support; wherein L¹ is a linker, and wherein TG is a terminating group, wherein preferably said terminating group is —H;
provided that X¹ and X² are not both —I.

In one aspect, the present disclosure provides a compound of the Formula I, or a pharmaceutically acceptable salt or solvate thereof:

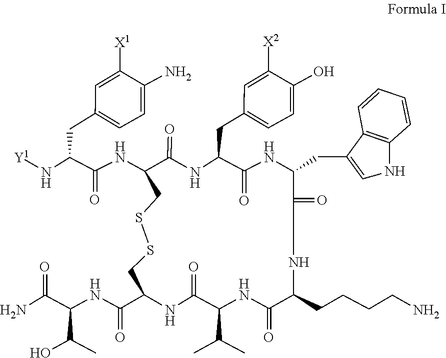

wherein:
X¹ is selected from the group consisting of —F, —Cl, —Br, and —I;
X² is selected from the group consisting of —F, —Cl, —Br, and —I; and
Y¹ is selected from the group consisting of —H, a chelator, L¹-TG, and -L¹-chelator; wherein L¹ is a linker, and wherein TG is a terminating group, wherein preferably said terminating group is —H;
provided that X¹ and X² are not both —I.

In some embodiments, the present disclosure provides a compound of the Formula I, or a pharmaceutically acceptable salt or solvate thereof:

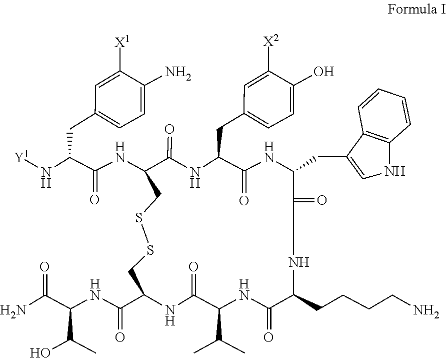

wherein:
X¹ is selected from the group consisting of —F, —Cl, —Br, and —I;
X² is selected from the group consisting of —F, —Cl, —Br, and —I; and
Y¹ is selected from the group consisting of —H, a chelator, L¹-H, and -L¹-chelator; wherein L¹ is a linker;
provided that X¹ and X² are not both —I.

In some embodiments, said compound of Formula I is of the Formula I-A, or a pharmaceutically acceptable salt or solvate thereof:

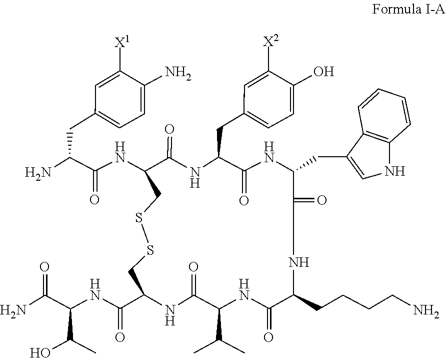

wherein:
X¹ is selected from the group consisting of —F, —Cl, —Br, and —I; and
X² is selected from the group consisting of —F, —Cl, —Br, and —I;
provided that X¹ and X² are not both —I.

In one aspect, the present invention provides a pharmaceutical composition comprising a compound of Formula I or Formula I-A and a pharmaceutically acceptable carrier. In one aspect, the present disclosure provides a pharmaceutical composition comprising a compound of Formula I and a pharmaceutically acceptable carrier. In one aspect, the present disclosure provides a pharmaceutical composition comprising a compound of Formula I-A and a pharmaceutically acceptable carrier.

In one aspect, the present invention provides a pharmaceutical composition comprising a compound of Formula I or Formula I-A, or a pharmaceutical composition comprising a compound of Formula I or Formula I-A for use as a medicament.

### DESCRIPTION OF FIGURES

FIG. 1A is a dose-response curve showing the percent activation of SST2 receptors effected by SS-28 as a function of SS-28 concentration. The dose-response curve was measured in CHO-K1 cells (Accession Number NP_001041.1) in the cAMP-HTRF assay as described in Example 11.

FIG. 1B is a dose-response curve showing the percent activation of SST2 receptors effected by Compound 3 as a function of Compound 3 concentration. The dose-response curve was measured in CHO-K1 cells (Accession Number NP_001041.1) in the cAMP-HTRF assay as described in Example 11.

FIG. 1C is a dose-response curve showing the percent activation of SST2 receptors effected by Compound 4 as a function of Compound 4 concentration. The dose-response curve was measured in CHO-K1 cells (Accession Number NP_001041.1) in the cAMP-HTRF assay as described in Example 11.

FIG. 1D is a dose-response curve showing the percent activation of SST2 receptors effected by Compound 5 as a function of Compound 5 concentration. The dose-response curve was measured in CHO-K1 cells (Accession Number NP_001041.1) in the cAMP-HTRF assay as described in Example 11.

FIG. 2A is a dose-response curve showing the results of a first competitive binding assay between ^{[125I]}SS-14 and SS-28 as a comparison to Compounds 1 and 2. The percent binding of ^{[125I]}SS-14 to SST2 receptors is shown as a function of SS-28 concentration. The dose-response curve was measured in CHO-K1 cells (Accession Number NP_001041.1) in the radioligand binding competition assay as described in Example 12.

FIG. 2B is a dose-response curve showing the results of the competitive binding assay between ^{[125I]}SS-14 and Compound 1. The percent binding of ^{[125I]}SS-14 to SST2 receptors is shown as a function of Compound 1 concentration. The dose-response curve was measured in CHO-K1 cells (Accession Number NP_001041.1) in the radioligand binding competition assay as described in Example 12.

FIG. 2C is a dose-response curve showing the results of the competitive binding assay between ^{[125I]}SS-14 and Compound 2. The percent binding of ^{[125I]}SS-14 to SST2 receptors is shown as a function of Compound 2 concentration. The dose-response curve was measured in CHO-K1 cells (Accession Number NP_001041.1) in the radioligand binding competition assay as described in Example 12.

FIG. 2D is a dose-response curve showing the results of a second competitive binding assay between ^{[125I]}SS-14 and SS-28 as a comparison to Compound 3. The percent binding of ^{[125I]}SS-14 to SST2 receptors is shown as a function of SS-28 concentration. The dose-response curve was measured in CHO-K1 cells (Accession Number NP_001041.1) in the radioligand binding competition assay as described in Example 12.

FIG. 2E is a dose-response curve showing the results of the competitive binding assay between ^{[125I]}SS-14 and Compound 3. The percent binding of ^{[125I]}SS-14 to SST2 receptors is shown as a function of Compound 3 concentration. The dose-response curve was measured in CHO-K1 cells (Accession Number NP_001041.1) in the radioligand binding competition assay as described in Example 12.

FIG. 2F is a dose-response curve showing the results of a third competitive binding assay between ^{[125I]}SS-14 and SS-28 as a comparison to Compounds 4 and 5. The percent binding of ^{[125I]}SS-14 to SST2 receptors is shown as a function of SS-28 concentration. The dose-response curve was measured in CHO-K1 cells (Accession Number NP_001041.1) in the radioligand binding competition assay as described in Example 12.

FIG. 2G is a dose-response curve showing the results of the competitive binding assay between ^{[125I]}SS-14 and Compound 4. The percent binding of ^{[125I]}SS-14 to SST2 receptors is shown as a function of Compound 4 concentration. The dose-response curve was measured in CHO-K1 cells (Accession Number NP_001041.1) in the radioligand binding competition assay as described in Example 12.

FIG. 2H is a dose-response curve showing the results of the competitive binding assay between ^{[125I]}SS-14 and Compound 5. The percent binding of ^{[125I]}SS-14 to SST2 receptors is shown as a function of Compound 5 concentration. The dose-response curve was measured in CHO-K1 cells (Accession Number NP_001041.1) in the radioligand binding competition assay as described in Example 12.

FIG. 3A is a dose-response curve showing the results of a first competitive binding assay between ^{[125I]}SS-14 and SS-28 as a comparison to Compounds 1 and 2. The percent binding of ^{[125I]}SS-14 to SST5 receptors is shown as a function of SS-28 concentration. The dose-response curve was measured in CHO-K1 cells (Accession Number NP_001044.1) in the radioligand binding competition assay as described in Example 12.

FIG. 3B is a dose-response curve showing the results of the competitive binding assay between ^{[125I]}SS-14 and Compound 1. The percent binding of ^{[125I]}SS-14 to SST5 receptors is shown as a function of Compound 1 concentration. The dose-response curve was measured in CHO-K1 cells (Accession Number NP_001044.1) in the radioligand binding competition assay as described in Example 12.

FIG. 3C is a dose-response curve showing the results of the competitive binding assay between ^{[125I]}SS-14 and Compound 2. The percent binding of ^{[125I]}SS-14 to SST5 receptors is shown as a function of Compound 2 concentration. The dose-response curve was measured in CHO-K1 cells (Accession Number NP_001044.1) in the radioligand binding competition assay as described in Example 12.

FIG. 3D is a dose-response curve showing the results of a second competitive binding assay between ^{[125I]}SS-14 and SS-28 as a comparison to Compound 3. The percent binding of ^{[125I]}SS-14 to SST5 receptors is shown as a function of SS-28 concentration. The dose-response curve was measured in CHO-K1 cells (Accession Number NP_001044.1) in the radioligand binding competition assay as described in Example 12.

FIG. 3E is a dose-response curve showing the results of the competitive binding assay between ^{[125I]}SS-14 and Compound 3. The percent binding of ^{[125I]}SS-14 to SST5 receptors is shown as a function of Compound 3 concentration. The dose-response curve was measured in CHO-K1 cells (Accession Number NP_001044.1) in the radioligand binding competition assay as described in Example 12.

FIG. 3F is a dose-response curve showing the results of a third competitive binding assay between ^{[125I]}SS-14 and SS-28 as a comparison to Compounds 4 and 5. The percent binding of ^{[125I]}SS-14 to SST5 receptors is shown as a function of SS-28 concentration. The dose-response curve was measured in CHO-K1 cells (Accession Number NP_001044.1) in the radioligand binding competition assay as described in Example 12.

FIG. 3G is a dose-response curve showing the results of the competitive binding assay between ^{[125I]}SS-14 and Compound 4. The percent binding of ^{[125I]}SS-14 to SST5 receptors is shown as a function of Compound 4 concentration. The dose-response curve was measured in CHO-K1 cells (Accession Number NP_001044.1) in the radioligand binding competition assay as described in Example 12.

FIG. 3H is a dose-response curve showing the results of the competitive binding assay between ^{[125I]}SS-14 and Compound 5. The percent binding of ^{[125I]}SS-14 to SST5 receptors is shown as a function of Compound 5 concentration. The dose-response curve was measured in CHO-K1 cells (Accession Number NP_001044.1) in the radioligand binding competition assay as described in Example 12.

FIG. 4A is a bar graph showing the change in ACTH levels secreted from AtT-20 cells after 72-hour incubation with Compound 1 at 5 nM and 10 nM. The change is shown normalized to untreated (control) cells.

FIG. 4B is a bar graph showing the change in ACTH levels secreted from AtT-20 cells after 72-hour incubation with Compound 2 at 5 nM and 10 nM. The change is shown normalized to untreated (control) cells.

FIG. 4C is a bar graph showing the change in ACTH levels secreted from AtT-20 cells after 72-hour incubation with Compound 3 at 5 nM and 10 nM. The change is shown normalized to untreated (control) cells.

FIG. 4D is a bar graph showing the change in ACTH levels secreted from AtT-20 cells after 72-hour incubation with Compound 4 at 5 nM and 10 nM. The change is shown normalized to untreated (control) cells.

FIG. 4E is a bar graph showing the change in ACTH levels secreted from AtT-20 cells after 72-hour incubation with Compound 5 at 5 nM and 10 nM. The change is shown normalized to untreated (control) cells.

FIG. 5A is a bar graph showing the change in GH levels secreted from GH3 cells after 72-hour incubation with Compound 1 at 5 nM and 10 nM. The change is shown normalized to untreated (control) cells.

FIG. 5B is a bar graph showing the change in GH levels secreted from GH3 cells after 72-hour incubation with Compound 2 at 5 nM and 10 nM. The change is shown normalized to untreated (control) cells.

FIG. 5C is a bar graph showing the change in GH levels secreted from GH3 cells after 72-hour incubation with Compound 3 at 5 nM and 10 nM. The change is shown normalized to untreated (control) cells.

FIG. 5D is a bar graph showing the change in GH levels secreted from GH3 cells after 72-hour incubation with Compound 4 at 5 nM and 10 nM. The change is shown normalized to untreated (control) cells.

FIG. 5E is a bar graph showing the change in GH levels secreted from GH3 cells after 72-hour incubation with Compound 5 at 5 nM and 10 nM. The change is shown normalized to untreated (control) cells.

FIG. 6A is a bar graph showing the change in insulin levels secreted from NT-3 cells after 5-day incubation with Compound 1 at 5 nM and 10 nM. The change is shown normalized to untreated (control) cells.

FIG. 6B is a bar graph showing the change in insulin levels secreted from NT-3 cells after 5-day incubation with Compound 2 at 5 nM and 10 nM. The change is shown normalized to untreated (control) cells.

FIG. 6C is a bar graph showing the change in insulin levels secreted from NT-3 cells after 5-day incubation with Compound 3 at 5 nM and 10 nM. The change is shown normalized to untreated (control) cells.

FIG. 6D is a bar graph showing the change in insulin levels secreted from NT-3 cells after 5-day incubation with Compound 4 at 5 nM and 10 nM. The change is shown normalized to untreated (control) cells.

FIG. 6E is a bar graph showing the change in insulin levels secreted from NT-3 cells after 5-day incubation with Compound 5 at 5 nM and 10 nM. The change is shown normalized to untreated (control) cells.

FIG. 7 is a bar graph showing the change in proliferation of NT-3 cells after 5-day incubation with Compounds 1, 4 and 5 at 5 nM. The change is shown normalized to untreated (control; NT) cells.

### DETAILED DESCRIPTION OF THE INVENTION

Unless defined otherwise, all technical and scientific terms used herein have the same meanings as commonly understood by one of ordinary skill in the art to which this invention belongs.

### Definitions

As used herein “a” or “an” means one or more, unless specifically indicated to mean only one.

The standard three-letter abbreviations identify the alpha-amino acid residues, and where the amino acid residue has isomeric forms, it is the L-form of the amino acid that is represented unless otherwise expressly indicated (e.g., Ser=L-serine). “L” or “D” refers to either of the D- and L-isomers of a particular amino acid.

The term “halogen” (or halo) represents fluoro (—F), chloro (—Cl), bromo (—Br), or iodo (—I).

“Administration” as used herein encompasses all suitable means of providing a substance to a subject. Common routes include oral, sublingual, transmucosal, transdermal, rectal, vaginal, subcutaneous, intramuscular, intravenous, intra-arterial, intrathecal, intra-articular, via catheter, via implant etc. Preferred routes in the context of the present invention are subcutaneous, oral, intramuscular, intravenous and intra-arterial. In some embodiments, a composition is administered near or directly to the tumor, such as by direct injection into the tumor or injection into the blood such as hematological malignancies.

“Subject” as used herein includes any vertebrate animal, including equine, ovine, caprine, bovine, porcine, avian, canine, feline and primate species. Preferably a “subject” as used herein is a human.

“Tumor” as used herein includes solid and non-solid tumors; and different stages of tumor development from pre-cancerous lesions (hyperplasia or dysplasia) and benign tumors (adenomas), to carcinoid, malignant and metastatic tumors. Preferably, the term “tumor” as used herein refers to any kind of abnormal growth of cells overexpressing one or more somatostatin receptors, in particular the somatostatin receptor subtypes 2 and 5 (SST2 and SST5). The term “neuroendocrine tumors” (NETs) refer to neoplasms that arise from cells of the endocrine and nervous system and can be either hormone-secreting or non-hormone-secreting.

The term “selectively binds” or “selective binding” herein refers to the preferential binding of the inventive compound(s) to particular somatostatin receptor subtypes, for example, the inventive compound(s) binds strongly to SST2 and SST5, while weakly or does not bind to other SSTs. Typically, and preferably, a “selective” compound of the present invention binds about 10 times, preferably about 100 times, or even more preferably about 1000 times (or more) more strongly to the selective somatostatin receptor subtypes than it does to other receptor subtypes, e.g., other somatostatin receptor subtypes.

As used herein, the term “effective amount” refers to an amount necessary or sufficient to realize a desired effect. Preferably, the term “effective amount” refers to an amount of a compound of Formula I or Formula I-A of the present invention that (i) treats or prevents the particular disease, medical condition, or disorder, (ii) attenuates, ameliorates, or eliminates one or more symptoms of the particular disease, medical condition, or disorder, or (iii) prevents or delays the onset of one or more symptoms of the particular disease, medical condition, or disorder described herein.

In preferred embodiments, therapeutically effective amounts of the inventive compounds are administered under the guidance of a physician, and pharmaceutical compositions thereof will contain the inventive compounds in conjunction with a conventional, pharmaceutically or veterinary acceptable carrier. A therapeutically effective amount is considered to be a predetermined amount calculated to achieve the desired effect. The required dosage will vary with the particular treatment and with the duration of desired treatment.

The terms “reducing” and “inhibiting” have their commonly understood meaning of lessening or decreasing.

The term “overexpression” is understood to mean expression of a protein beyond the amount of expression found in a normal, healthy cell.

The terms “pharmaceutically acceptable” or “therapeutically acceptable” refers to a substance which does not interfere with the effectiveness or the biological activity of the active ingredients and which is not toxic to the host. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, hydroiodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. Preferred pharmaceutically acceptable salts include hydrochloride, hydrobromide, sulphate, phosphate, tannate, oxalate, fumarate, gluconate, alginate, maleate, acetate, citrate, benzoate, succinate, malate, ascorbate, tartrate and the like, preferably acetate.

A “solvate” refers to an association or complex of one or more solvent molecules and a compound of the present invention. Examples of solvents that form solvates include, but are not limited to, water, isopropanol, ethanol, methanol, dimethyl sulfoxide (DMSO), ethyl acetate, acetic acid, and ethanolamine. The term “hydrate” refers to the complex where the solvent molecule is water.

A “radionuclide” as used herein refers to an unstable form of an atomic nucleus that has excess nuclear energy. Preferably a radionuclide as used herein releases the excess nuclear energy (e.g., as radiation) as it breaks down into more stable nuclides. In some embodiments, the radionuclide is selected from the group consisting of boron-10, fluorine-18, phosphorus-32, scandium-47, copper-67, gallium-72, rubidium-82, strontium-89, yttrium-90, technetium-99, palladium-103, indium-111, iodine-125, caesium-131, iodine-131, samarium-153, gadolinium-157, gadolinium-159, terbium-149, samarium-153, terbium-161, dysprosium-165, holmium-166, ytterbium-175, lutetium-177, rhenium-186, rhenium-188, thallium-201, astatine-211, lead-212, bismuth-212, bismuth-213, radium-223, actinium-225, and thorium-227.

A “chelator” as used herein refers to a chemical moiety, preferably an organic moiety, capable of forming coordinate, preferably polydentate bonds to a metal atom (preferably a metal ion). In preferred embodiments a chelator can form a stable complex (e.g., a coordinate polydentate complex) with a radionuclide.

A “linker” as used herein refers to a bivalent organic residue, wherein one of the valencies is bonded to a compound of Formula I, and the other valency is bonded to a terminating group (abbreviated herein as “TG”) or a chelator, wherein preferably said terminating group is —H.

As used herein, a “solid support” includes without a limitation surfaces, beads, glass supports, polymers or resins. In a preferred embodiment, the glass is controlled-pore glass, preferably with 500 Å, 1000 Å or 2000 Å pores. The beads include without limitation glass beads, preferably controlled-pore glass, or magnetic beads. The polymer includes without limitation polystyrenes including for example divinylbenzene, styrene, and chloromethylstyrene. In a preferred embodiment, the solid support are highly cross-linked polystyrene beads.

The term “amino protecting group” is well known in the art and includes those described in detail in Protecting Groups in Organic Synthesis, T. W. Greene and P. G. M. Wuts, 3^{rd} edition, John Wiley & Sons, 1999, Greene's Protective Groups in Organic Synthesis, P. G. M. Wuts, 5^{th} edition, John Wiley & Sons, 2014, and in Current Protocols in Nucleic Acid Chemistry, edited by S. L. Beaucage et al. 06/2012, and hereby in particular in Chapter 2. Suitable “amino protecting groups” for the present invention include and are typically and preferably independently at each occurrence selected from methyl carbamate, ethyl carbamate, 9-fluorenylmethyl carbamate (Fmoc), 9-(2-sulfo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1,1-dimethyl-2,2-dibromoethyl carbamate (DB-t-BOC), 1,1-dimethyl-2,2,2-trichloroethyl carbamate (TCBOC), benzyl carbamate (Cbz), p-methoxybenzyl carbamate (Moz) and 2,4,6-trimethylbenzyl carbamate; as well as formamide, acetamide, benzamide.

### Compounds of the Invention

In one aspect, the present disclosure provides a compound of the Formula I, or a pharmaceutically acceptable salt or solvate thereof:

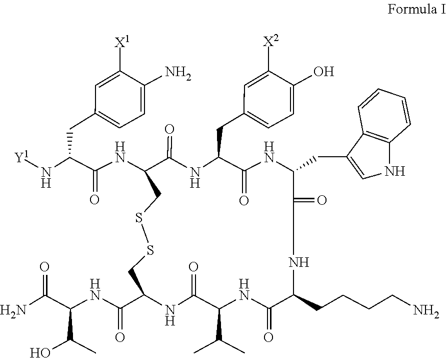

wherein:
X¹ is selected from the group consisting of —F, —Cl, —Br, and —I;
X² is selected from the group consisting of —F, —Cl, —Br, and —I; and
Y¹ is selected from the group consisting of —H, a chelator, L¹-TG, -L¹-chelator, an amino protecting group, and a solid support; wherein L¹ is a linker, and wherein TG is a terminating group, wherein preferably said terminating group is —H;
provided that X¹ and X² are not both —I.

In one aspect, the present disclosure provides a compound of the Formula I, or a pharmaceutically acceptable salt or solvate thereof:

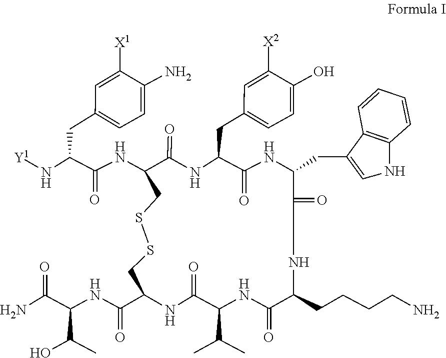

wherein:
X¹ is selected from the group consisting of —F, —Cl, —Br, and —I;
X² is selected from the group consisting of —F, —Cl, —Br, and —I; and
Y¹ is selected from the group consisting of —H, a chelator, L¹-TG, and -L¹-chelator; wherein L¹ is a linker, and wherein TG is a terminating group, wherein preferably said terminating group is —H;
provided that X¹ and X² are not both —I.

In some embodiments, the present disclosure provides a compound of the Formula I, or a pharmaceutically acceptable salt or solvate thereof:

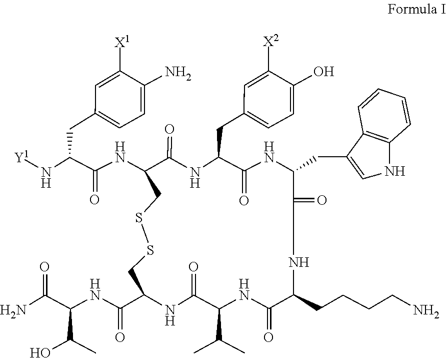

wherein:
X¹ is selected from the group consisting of —F, —Cl, —Br, and —I;
X² is selected from the group consisting of —F, —Cl, —Br, and —I; and
Y¹ is selected from the group consisting of —H, a chelator, L¹-H, and -L¹-chelator; wherein L¹ is a linker;
provided that X¹ and X² are not both —I.

In some embodiments, Y¹ is selected from the group consisting of —H, a chelator, L¹-TG, -L¹-chelator, and an amino protecting group; wherein L¹ is a linker, and wherein TG is a terminating group, wherein preferably said terminating group is —H;

In some embodiments, Y¹ is selected from the group consisting of —H, a chelator, L¹-TG, -L¹-chelator, and a solid support; wherein L¹ is a linker, and wherein TG is a terminating group, wherein preferably said terminating group is —H;

In some embodiments, Y¹ is selected from the group consisting of —H, a chelator, L¹-TG, and -L¹-chelator; wherein L¹ is a linker, and wherein TG is a terminating group, wherein preferably said terminating group is —H;

In some embodiments, Y¹ is selected from the group consisting of a chelator and -L¹-chelator. In some embodiments, Y¹ is -L¹-chelator. In some embodiments, Y₁ is a chelator. In some embodiments, Y¹ is L¹-TG. In some embodiments, Y¹ is L¹-H. In some embodiments, Y¹ is an amino protecting group. In some embodiments, Y¹ is a solid support. In some embodiments, Y¹ is —H.

In some embodiments, said chelator is complexed to a radionuclide. In preferred embodiments, said radionuclide is selected from the group consisting of boron-10, fluorine-18, phosphorus-32, scandium-47, copper-67, gallium-72, rubidium-82, strontium-89, yttrium-90, technetium-99, palladium-103, indium-111, iodine-125, caesium-131, iodine-131, samarium-153, gadolinium-157, gadolinium-159, terbium-149, samarium-153, terbium-161, dysprosium-165, holmium-166, ytterbium-175, lutetium-177, rhenium-186, rhenium-188, thallium-201, astatine-211, lead-212, bismuth-212, bismuth-213, radium-223, actinium-225, and thorium-227.

In some embodiments, said compound of Formula I is of the Formula I-A, or a pharmaceutically acceptable salt or solvate thereof:

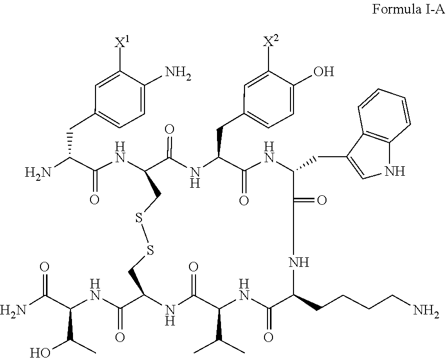

wherein:
X¹ is selected from the group consisting of —F, —Cl, —Br, and —I; and
X² is selected from the group consisting of —F, —Cl, —Br, and —I;
provided that X¹ and X² are not both —I.

In one aspect, the present invention provides a pharmaceutical composition comprising a compound of Formula I or Formula I-A and a pharmaceutically acceptable carrier.

In some embodiments of Formula I or Formula I-A, X¹ and X² are the same and are both selected from the group consisting of —F, —Cl, and —Br. In some embodiments of Formula I or Formula I-A, X¹ and X² are the same and are both selected from the group consisting of —Cl and —Br. In some embodiments, X¹ and X² are the same and are both selected from the group consisting of —Cl and —F. In some embodiments, X¹ and X² are the same and are both selected from the group consisting of —F and —Br.

In some embodiments of Formula I or Formula I-A, X¹ is —I and X² is —Cl; X¹ is —Cl and X² is —I; X¹ is —Cl and X² is —Cl; X¹ is —F and X² is —F; or X¹ is —Br and X² is —Br.

In some embodiments of Formula I or Formula I-A, X¹ is —I and X² is —Cl. In some embodiments, the compound of Formula I or Formula I-A is Compound 1:

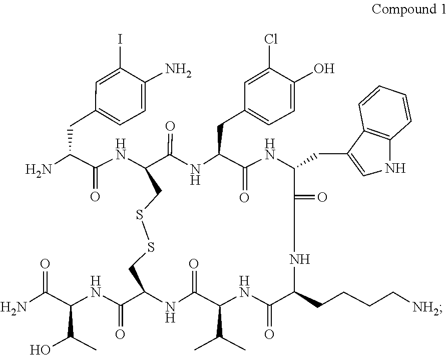 [SMILES: CC(C)[C@@H]1NC(=O)[C@H](CCCCN)NC(=O)[C@@H](Cc2c[nH]c3ccccc23)NC(=O)[C@H](Cc2ccc(O)c(Cl)c2)NC(=O)[C@H](NC(=O)[C@H](N)Cc2ccc(N)c(I)c2)CSSC[C@H](C(=O)N[C@H](C(N)=O)C(C)O)NC1=O]

or a pharmaceutically acceptable salt or solvate thereof.

In some embodiments of Formula I or Formula I-A, X¹ is —Cl and X² is —I. In some embodiments, the compound of Formula I or Formula I-A is Compound 2:

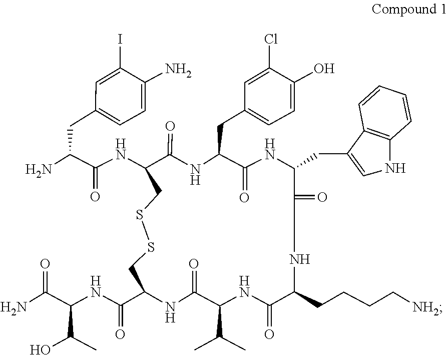 [SMILES: CC(C)[C@@H]1NC(=O)[C@H](CCCCN)NC(=O)[C@@H](Cc2c[nH]c3ccccc23)NC(=O)[C@H](Cc2ccc(O)c(Cl)c2)NC(=O)[C@H](NC(=O)[C@H](N)Cc2ccc(N)c(I)c2)CSSC[C@H](C(=O)N[C@H](C(N)=O)C(C)O)NC1=O]

or a pharmaceutically acceptable salt or solvate thereof.

In some embodiments of Formula I or Formula I-A, X¹ is —Cl and X² is —Cl. In some embodiments, the compound of Formula I or Formula I-A is Compound 3:

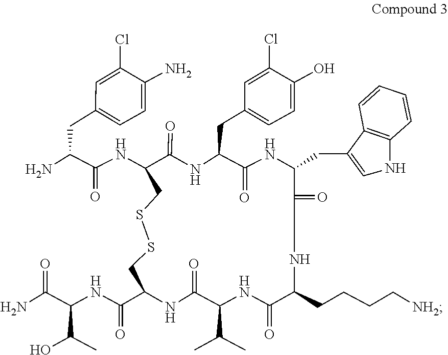 [SMILES: CC(C)[C@@H]1NC(=O)[C@H](CCCCN)NC(=O)[C@@H](Cc2c[nH]c3ccccc23)NC(=O)[C@H](Cc2ccc(O)c(Cl)c2)NC(=O)[C@H](NC(=O)[C@H](N)Cc2ccc(N)c(Cl)c2)CSSC[C@H](C(=O)N[C@H](C(N)=O)C(C)O)NC1=O]

or a pharmaceutically acceptable salt or solvate thereof.

In some embodiments of Formula I or Formula I-A, X¹ is —F and X² is —F. In some embodiments, the compound of Formula I or Formula I-A is Compound 4:

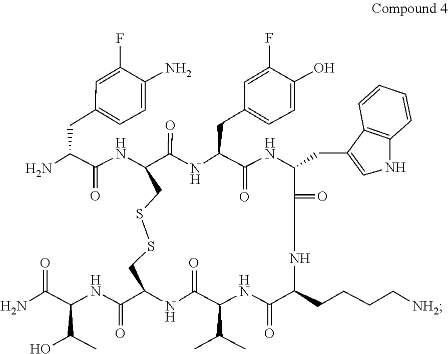 [SMILES: CC(C)[C@@H]1NC(=O)[C@H](CCCCN)NC(=O)[C@@H](Cc2c[nH]c3ccccc23)NC(=O)[C@H](Cc2ccc(O)c(F)c2)NC(=O)[C@H](NC(=O)[C@H](N)Cc2ccc(N)c(F)c2)CSSC[C@H](C(=O)N[C@H](C(N)=O)C(C)O)NC1=O]

or a pharmaceutically acceptable salt or solvate thereof.

In some embodiments of Formula I or Formula I-A, X¹ is —Br and X² is —Br. In some embodiments, the compound of Formula I or Formula I-A is Compound 5:

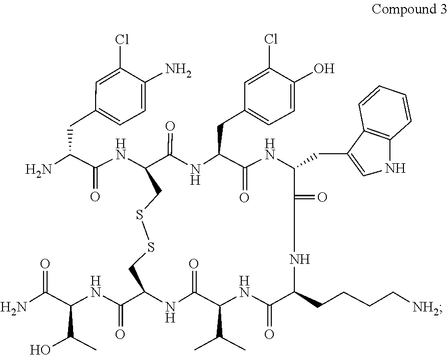 [SMILES: CC(C)[C@@H]1NC(=O)[C@H](CCCCN)NC(=O)[C@@H](Cc2c[nH]c3ccccc23)NC(=O)[C@H](Cc2ccc(O)c(Cl)c2)NC(=O)[C@H](NC(=O)[C@H](N)Cc2ccc(N)c(Cl)c2)CSSC[C@H](C(=O)N[C@H](C(N)=O)C(C)O)NC1=O]

or a pharmaceutically acceptable salt or solvate thereof.

In some embodiments, the compound of Formula I is selected from the group consisting of:

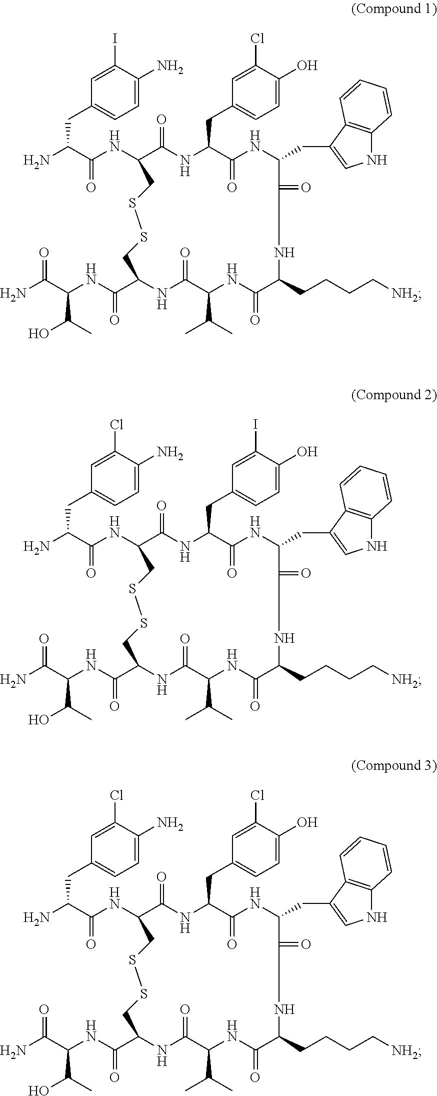

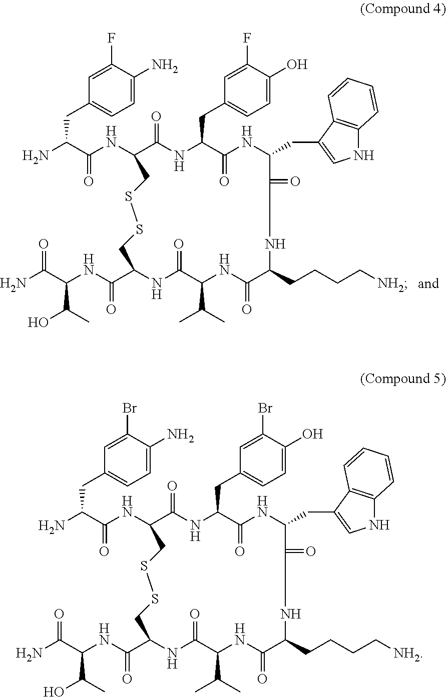 [SMILES: CC(C)[C@@H]1NC(=O)[C@H](CCCCN)NC(=O)[C@@H](Cc2c[nH]c3ccccc23)NC(=O)[C@H](Cc2ccc(O)c(F)c2)NC(=O)[C@H](NC(=O)[C@H](N)Cc2ccc(N)c(F)c2)CSSC[C@H](C(=O)N[C@H](C(N)=O)C(C)O)NC1=O ; "and" ; CC(C)[C@@H]1NC(=O)[C@H](CCCCN)NC(=O)[C@@H](Cc2c[nH]c3ccccc23)NC(=O)[C@H](Cc2ccc(O)c(Br)c2)NC(=O)[C@H](NC(=O)[C@H](N)Cc2ccc(N)c(Br)c2)CSSC[C@H](C(=O)N[C@H](C(N)=O)C(C)O)NC1=O]

In one aspect, the present invention provides a pharmaceutical composition comprising a compound of Formula I or Formula I-A and a pharmaceutically acceptable carrier. In one aspect, the present disclosure provides a pharmaceutical composition comprising a compound of Formula I and a pharmaceutically acceptable carrier. In one aspect, the present disclosure provides a pharmaceutical composition comprising a compound of Formula I-A and a pharmaceutically acceptable carrier.

### Use of the Inventive Compounds

The inventive compounds bind to somatostatin receptors (e.g., SST2 and/or SST5) with high affinity and selectivity and can therefore be used in the treatment of diseases such as hormone excess, proliferative diseases, cancer (e.g., neuroendocrine cancer) and/or a tumor as described herein.

In preferred embodiments, the inventive compounds bind to and activate somatostatin receptors (e.g., SST2 and/or SST5); preferably the inventive compounds selectively bind to and activate SST2 and/or SST5. In preferred embodiments, the inventive compounds are internalized by cells after binding to the SST2 and/or SST5 receptors.

Example 11 demonstrates that the inventive compounds bound to SST2 with sub- and low-nanomolar EC₅₀ values in a cAMP HTRF assay. The binding affinity of the inventive compounds to SST2 receptor was comparable to that of SS-28, a natural ligand of SST2. Without wishing to be bound by theory, halogenation of the inventive compounds at positions X¹ and X² did not appreciably diminish binding of the inventive compounds to the SST2 receptor.

As described in Example 12, the inventive compounds were tested in a radioligand binding competition assay against ^{[125I]}SS-14 in membrane extracts from CHO-K1 cells expressing either SST2 or SST5. Assays were performed on different days using SS-28 as a standard for comparison to evaluate the ability of the inventive compounds to bind SST2 and SST5. In the first assay, Compounds 1 and 2 were tested in the radioligand binding competition assay against ^{[125I]}SS-14 and SS-28 was evaluated in parallel as a standard for comparison. In the second assay, Compound 3 was tested against ^{[125I]}SS-14 and SS-28 was evaluated in parallel. In the third assay, Compounds 4 and 5 were tested against ^{[125I]}SS-14 and SS-28 was evaluated in parallel.

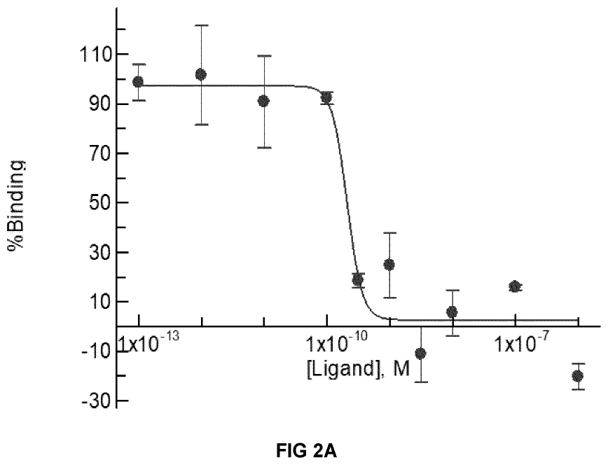

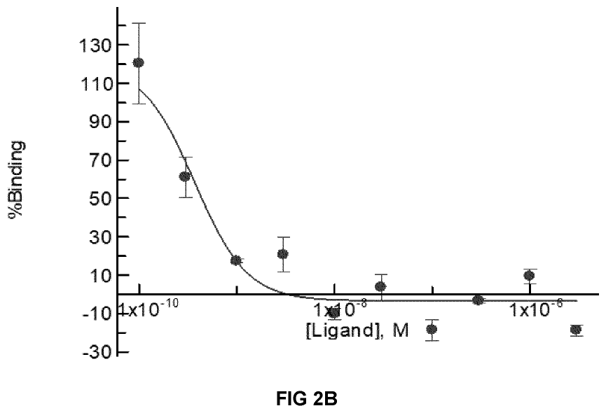

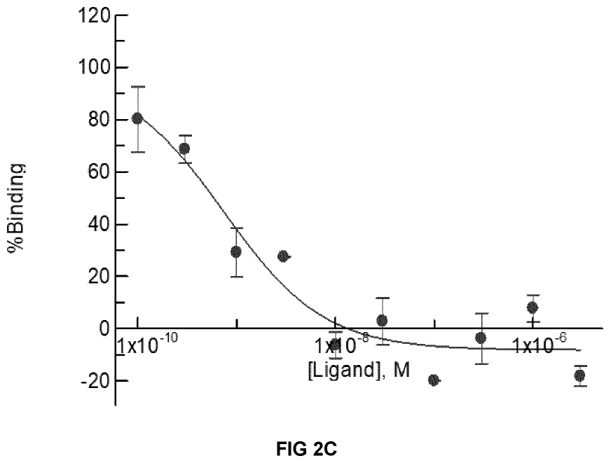

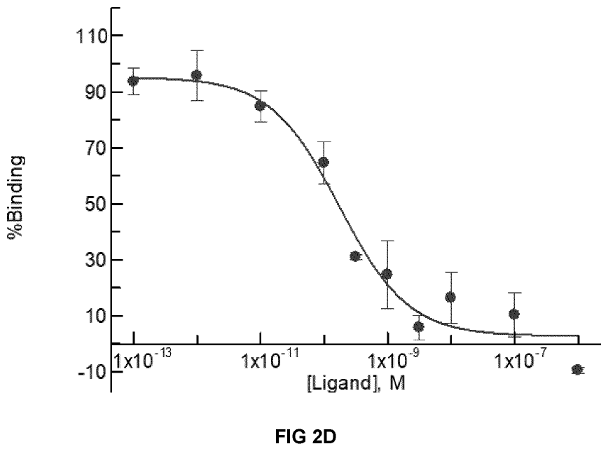

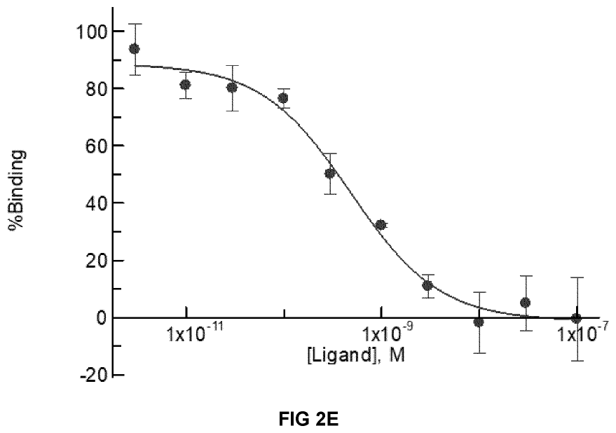

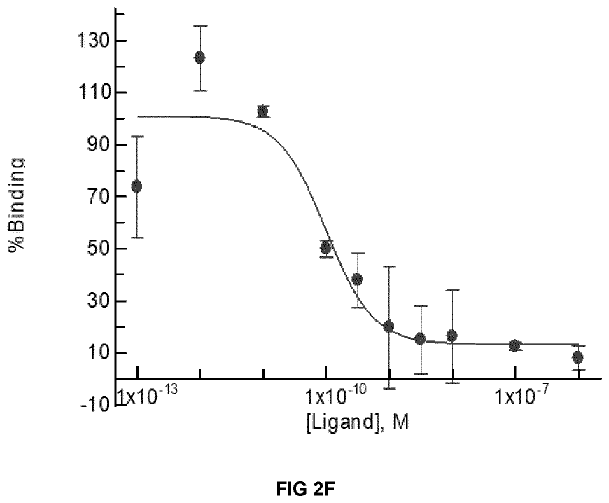

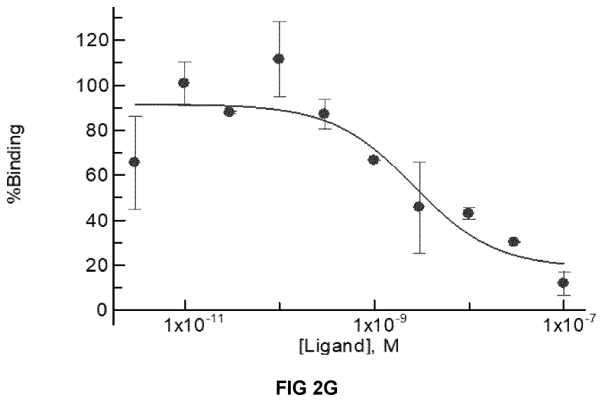

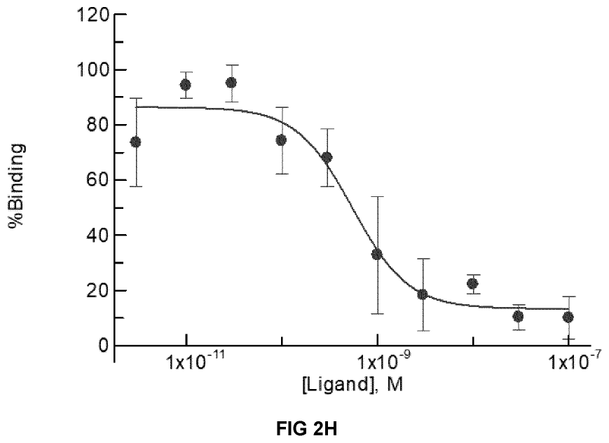

As shown in Example 12, Tables 2-4 and FIGS. 2A-2H, the inventive compounds exhibited sub- and low-nanomolar binding affinity to SST2 receptors in a radioligand binding competition assay. The binding affinity of the inventive compounds was comparable to that of SS-28, a natural ligand of SST2. Without wishing to be bound by theory, halogenation of the inventive compounds at positions X¹ and X² did not appreciably diminish binding of the inventive compounds to the SST2 receptor.

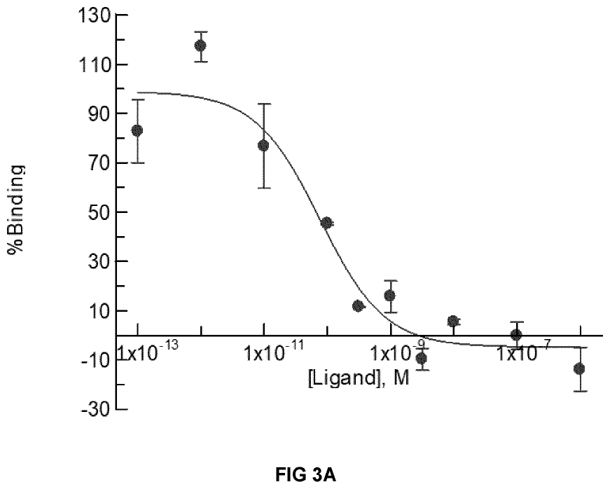

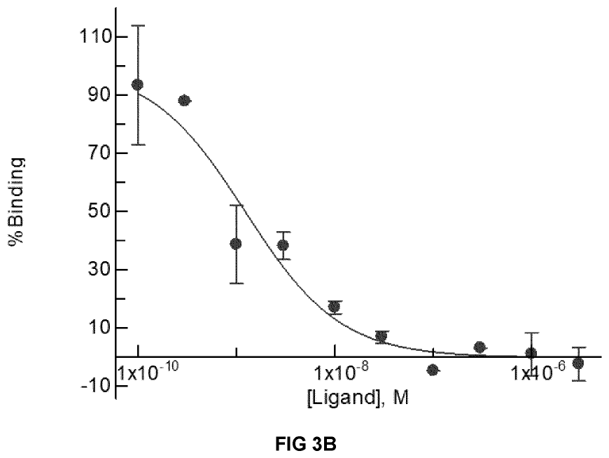

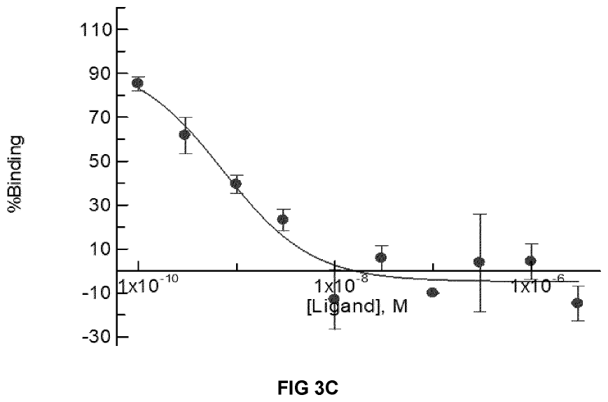

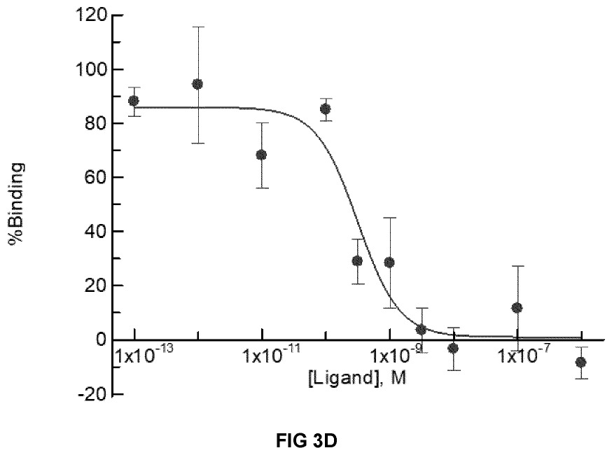

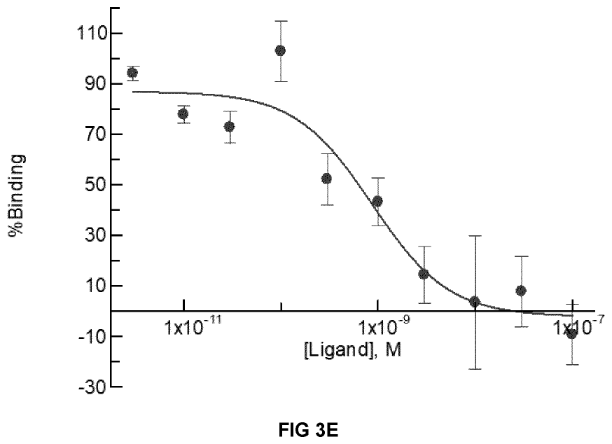

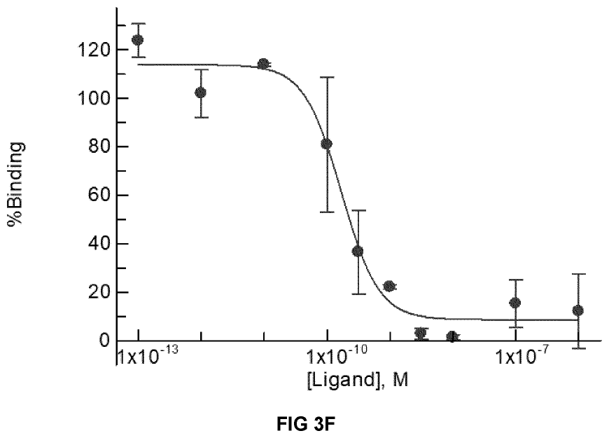

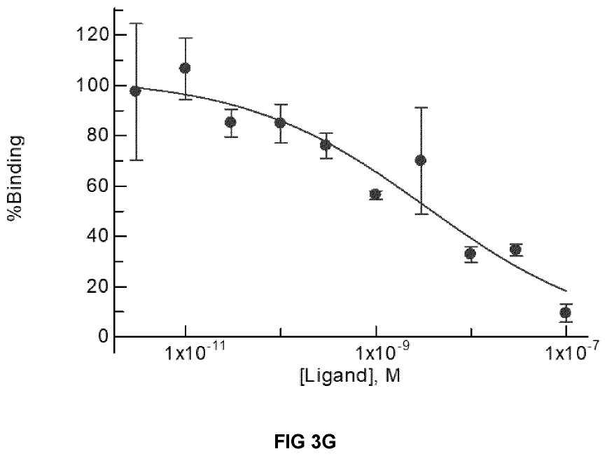

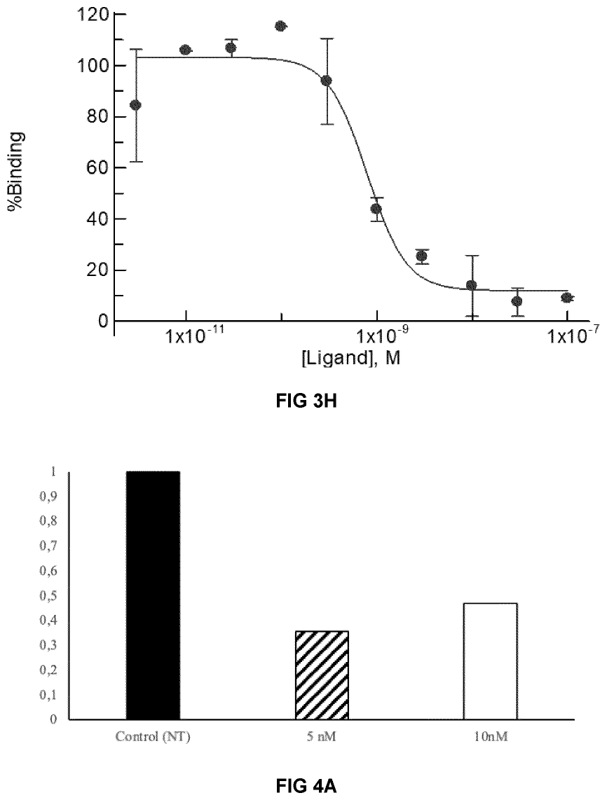

As shown in Example 12, Tables 5-7 and FIGS. 3A-3H, the inventive compounds exhibited sub- and low-nanomolar binding affinity to SST5 receptors in a radioligand binding competition assay. The binding affinity of the inventive compounds was comparable to that of SS-28, a natural ligand of SST5. Without wishing to be bound by theory, halogenation of the inventive compounds at positions X¹ and X² did not appreciably diminish binding of the inventive compounds to the SST5 receptor.

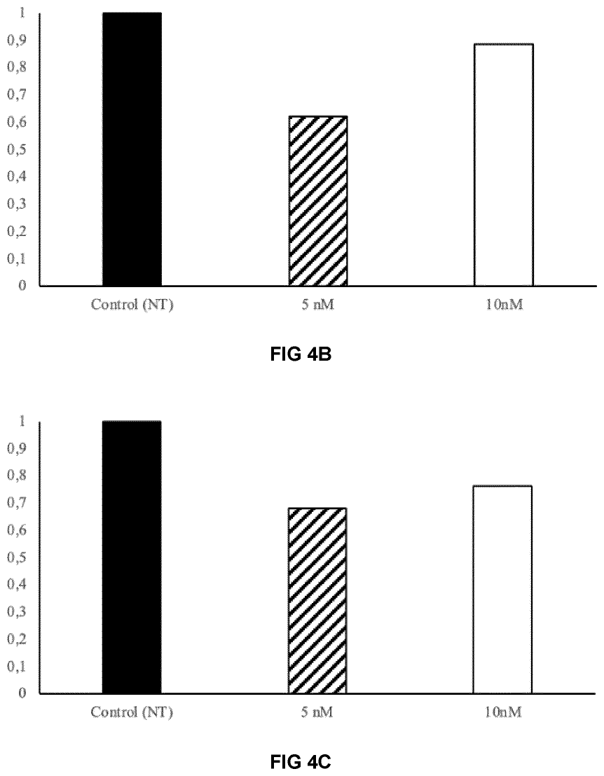

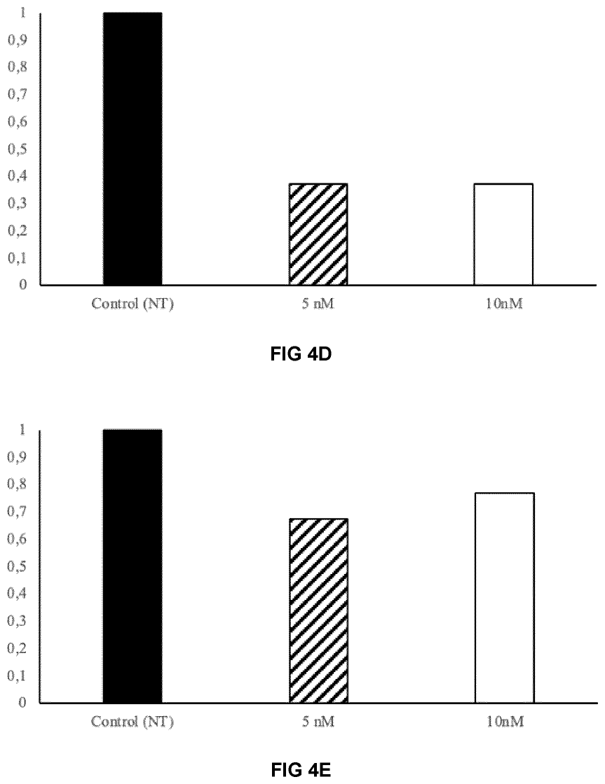

As shown in Examples 13-14 and FIGS. 4A-4E, 5A-5E and 6A-6E, the inventive compounds inhibited release of hormones such as adrenocorticotropin (ACTH), growth hormone (GH), and insulin.

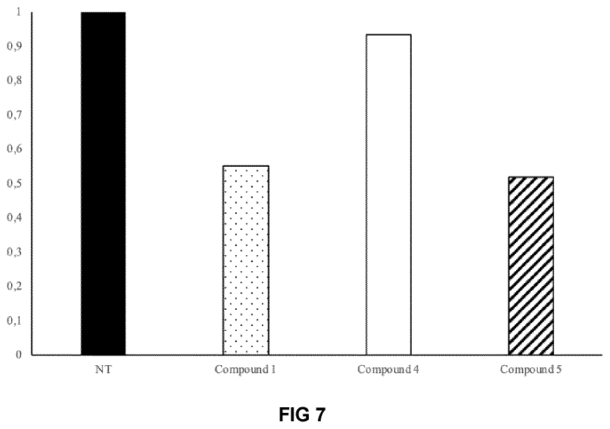

As shown in Example 16 and FIG. 7, the inventive compounds inhibited proliferation of human tumor cells.

In one aspect, the present invention provides a compound of Formula I or Formula I-A, or a pharmaceutical composition comprising a compound of Formula I or Formula I-A for use as a medicament.

In one aspect, the present invention provides a compound of Formula I or Formula I-A or a pharmaceutical composition comprising a compound of Formula I or Formula I-A, for use in a method of treating a SST2 or a SST5 cell proliferative disorder, preferably a proliferative disorder involving cells expressing SST2 and SST5, in a subject, e.g., a subject in need thereof, and preferably evaluating the subject after treatment with said compound or pharmaceutical composition, wherein further preferably said cell proliferative disorder is a tumor, e.g., a primary tumor or a metastatic tumor. In preferred embodiments, said tumor is a neuroendocrine tumor, preferably a neuroendocrine tumor of the pituitary, pancreas, gastrointestinal tract, lung, thymus, breast, prostate, and/or adrenal gland. In some embodiments, said proliferative disorder is thyroid cancer, melanoma, meningioma, primary brain cancer, salivary gland cancer, ovarian cancer, and/or hepatocellular carcinoma.

In a further aspect, the present invention provides a method of treating a SST2 or a SST5 cell proliferative disorder, preferably a proliferative disorder involving cells expressing SST2 and SST5, in a subject in need thereof, the method comprising administering to said subject an effective amount of a compound of Formula I or Formula I-A or a pharmaceutical composition comprising a compound of Formula I or Formula I-A; and preferably evaluating the subject after treatment with said compound or pharmaceutical composition, wherein further preferably said cell proliferative disorder is a tumor, e.g., a primary tumor or a metastatic tumor. In preferred embodiments, said tumor is a neuroendocrine tumor, preferably a neuroendocrine tumor of the pituitary, pancreas, gastrointestinal tract, lung, thymus, breast, prostate, and/or adrenal gland. In some embodiments, said proliferative disorder is thyroid cancer, melanoma, meningioma, primary brain cancer, salivary gland cancer, ovarian cancer, and/or hepatocellular carcinoma. In a further aspect, the present invention provides the use of a compound of Formula I or Formula I-A in the manufacture of a medicament for treatment of a SST2 or a SST5 cell proliferative disorder, preferably a proliferative disorder involving cells expressing SST2 and SST5; and preferably evaluating the subject after treatment with said compound or pharmaceutical composition, wherein further preferably said cell proliferative disorder is a tumor, e.g., a primary tumor or a metastatic tumor. In preferred embodiments, said tumor is a neuroendocrine tumor, preferably a neuroendocrine tumor of the pituitary, pancreas, gastrointestinal tract, lung, thymus, breast, prostate, and/or adrenal gland. In some embodiments, said proliferative disorder is thyroid cancer, melanoma, meningioma, primary brain cancer, salivary gland cancer, ovarian cancer, and/or hepatocellular carcinoma.

In a further aspect, the present invention provides a compound of Formula I or Formula I-A or a pharmaceutical composition comprising a compound of Formula I or Formula I-A for use in a method of treating a disease that is characterized by an overexpression of one or more somatostatin receptor subtypes, preferably by an overexpression of SST2 and/or SST5, more preferably by an overexpression of SST2 and SST5, in a subject, e.g., a subject in need thereof.

In a further aspect, the present invention provides a method of treating a disease that is characterized by an overexpression of one or more somatostatin receptor subtypes, preferably by an overexpression of SST2 and/or SST5, more preferably by an overexpression of SST2 and SST5, in a subject in need thereof; the method comprising administering to the subject an effective amount of a compound of Formula I or Formula I-A or a pharmaceutical composition comprising a compound of Formula I or Formula I-A.

In a further aspect, the present invention provides the use of a compound of Formula I or Formula I-A in the manufacture of a medicament for the treatment of a disease that is characterized by an overexpression of one or more somatostatin receptor subtypes, preferably by an overexpression of SST2 and/or SST5, more preferably by an overexpression of SST2 and SST5.

In a preferred embodiment, said SST2 or SST5 cell proliferative disorder, or said disease that is characterized by an overexpression of one or more somatostatin receptor subtypes is a tumor, preferably a neuroendocrine tumor.

In a further aspect, the present disclosure provides a compound of Formula I or Formula I-A, or a pharmaceutical composition comprising a compound of Formula I or Formula I-A for use in a method of treating a tumor, preferably a neuroendocrine tumor, preferably wherein said method comprises administering an effective amount of said compound or said pharmaceutical composition to a subject, e.g., a subject in need thereof. In preferred embodiments, the method further comprises evaluating the subject after treatment with said compound or said pharmaceutical composition.

In one aspect, the present disclosure provides a method of treating a tumor, preferably a neuroendocrine tumor, in a subject in need thereof, the method comprising administering to said subject an effective amount of a compound of Formula I or Formula I-A, or a pharmaceutical composition comprising a compound of Formula I or Formula I-A. In preferred embodiments, the method further comprises evaluating the subject after treatment with said compound or said pharmaceutical composition.

In one aspect, the present disclosure provides the use of a compound of Formula I or Formula I-A in the manufacture of a medicament for the treatment of a tumor, preferably a neuroendocrine tumor.

In a further aspect, the present invention provides a compound of Formula I or Formula I-A, or a pharmaceutical composition comprising compound of Formula I or Formula I-A, for use in a method of reducing the activity and growth of a tumor, preferably of a neuroendocrine tumor, in a subject, e.g., a subject in need thereof; and preferably evaluating the subject after treatment with said compound or pharmaceutical composition.

In a further aspect, the present invention provides a method of reducing the activity and growth of a tumor, preferably of a neuroendocrine tumor, in a subject in need thereof comprising administering to the subject an effective amount of a compound of Formula I or Formula I-A, or a pharmaceutical composition comprising compound of Formula I or Formula I-A; and preferably evaluating the subject after treatment with said compound or pharmaceutical composition.

In a further aspect, the present invention provides the use of a compound of Formula I or Formula I-A in the manufacture of a medicament for reducing the activity and growth of a tumor, preferably of a neuroendocrine tumor.

In a further aspect, the present invention provides a compound of Formula I or Formula I-A or a pharmaceutical composition comprising a compound of Formula I or Formula I-A for use in a method of reducing the release of a hormone or neurotransmitter, preferably growth hormone (GH), prolactin (PRL), thyroid stimulating hormone (TSH), adrenocorticotropic hormone (ACTH), gastrin, insulin, insulin-like growth factor 1 (IGF-1), glucagon, vasoactive intestinal polypeptide (VIP), pancreatic polypeptide, calcitonin, somatostatin, serotonin, epinephrine, norepinephrine, or dopamine in a subject, e.g., a subject in need thereof: preferably wherein said GH, PRL, TSH and/or ACTH is released from the anterior pituitary of the subject and wherein said gastrin, insulin, insulin-like growth factor 1 (IGF-1), glucagon, vasoactive intestinal polypeptide (VIP), pancreatic polypeptide, calcitonin, somatostatin, serotonin, epinephrine, norepinephrine, or dopamine is released by neuroendocrine cells in the pancreas, gastrointestinal tract, liver, lung, thyroid, parathyroid, hypothalamus, adrenal glands or sympathetic and parasympathetic ganglia, of the subject, e.g., a subject in need thereof; and preferably evaluating the subject after treatment with said compound or pharmaceutical composition.

In a further aspect, the present invention provides a method of reducing the release of a hormone or neurotransmitter in a subject in need thereof, the method comprising administering to said subject a compound of Formula I or Formula I-A or a pharmaceutical composition comprising a compound of Formula I or Formula I-A, preferably wherein said hormone or neurotransmitter is growth hormone (GH), prolactin (PRL), thyroid stimulating hormone (TSH), adrenocorticotropic hormone (ACTH), gastrin, insulin, insulin-like growth factor 1 (IGF-1), glucagon, vasoactive intestinal polypeptide (VIP), pancreatic polypeptide, calcitonin, somatostatin, serotonin, epinephrine, norepinephrine, or dopamine; further preferably wherein said GH, PRL, TSH and/or ACTH is released from the anterior pituitary of the subject and wherein said gastrin, insulin, insulin-like growth factor 1 (IGF-1), glucagon, vasoactive intestinal polypeptide (VIP), pancreatic polypeptide, calcitonin, somatostatin, serotonin, epinephrine, norepinephrine, or dopamine is released by neuroendocrine cells in the pancreas, gastrointestinal tract, liver, lung, thyroid, parathyroid, hypothalamus, adrenal glands or sympathetic and parasympathetic ganglia, of the subject, e.g., a subject in need thereof; and preferably evaluating the subject after treatment with said compound or pharmaceutical composition.

In a further aspect, the present invention provides the use of a compound of Formula I or Formula I-A in the manufacture of a medicament for reducing the release of a hormone or neurotransmitter in a subject in need thereof, preferably wherein said hormone or neurotransmitter is growth hormone (GH), prolactin (PRL), thyroid stimulating hormone (TSH), adrenocorticotropic hormone (ACTH), gastrin, insulin, insulin-like growth factor 1 (IGF-1), glucagon, vasoactive intestinal polypeptide (VIP), pancreatic polypeptide, calcitonin, somatostatin, serotonin, epinephrine, norepinephrine, or dopamine; further preferably wherein said GH, PRL, TSH and/or ACTH is released from the anterior pituitary of the subject and wherein said gastrin, insulin, insulin-like growth factor 1 (IGF-1), glucagon, vasoactive intestinal polypeptide (VIP), pancreatic polypeptide, calcitonin, somatostatin, serotonin, epinephrine, norepinephrine, or dopamine is released by neuroendocrine cells in the pancreas, gastrointestinal tract, liver, lung, thyroid, parathyroid, hypothalamus, adrenal glands or sympathetic and parasympathetic ganglia, of the subject, e.g., a subject in need thereof; and preferably additionally evaluating the subject after treatment with said compound or pharmaceutical composition.

In some embodiments, the compounds of the invention inhibit the release of growth hormone. In some embodiments, the compounds of the invention inhibit the release of thyroid stimulating hormone. In some embodiments, the compounds of the invention inhibit the release of prolactin. In some embodiments, the compounds of the invention inhibit the release of adrenocorticotropic hormone. In some embodiments, the compounds of the invention inhibit the release of gastrin. In some embodiments, the compounds of the invention inhibit the release of insulin. In some embodiments, the compounds of the invention inhibit the release of insulin-like growth factor 1 (IGF-1). In some embodiments, the compounds of the invention inhibit the release of glucagon. In some embodiments, the compounds of the invention inhibit the release of vasoactive intestinal polypeptide (VIP). In some embodiments, the compounds of the invention inhibit the release of pancreatic polypeptide. In some embodiments, the compounds of the invention inhibit the release of calcitonin. In some embodiments, the compounds of the invention inhibit the release of somatostatin. In some embodiments, the compounds of the invention inhibit the release of serotonin. In some embodiments, the compounds of the invention inhibit the release of epinephrine. In some embodiments, the compounds of the invention inhibit the release of norepinephrine. In some embodiments, the compounds of the invention inhibit the release of dopamine.

In some embodiments, said disease or disorder associated with excess secretion of a hormone, e.g., growth hormone and/or insulin-like growth factor 1, is selected from acromegaly and pituitary gigantism.

In a further aspect, the present invention provides a compound of Formula I or Formula I-A or a pharmaceutical composition comprising a compound of Formula I or Formula I-A for use in a method of decreasing blood flow in a subject, e.g., a subject in need thereof.

In a further aspect, the present invention provides a method of decreasing blood flow in a subject in need thereof, the method comprising administering to the subject an effective amount of a compound of Formula I or Formula I-A or a pharmaceutical composition comprising a compound of Formula I or Formula I-A.

In a further aspect, the present invention provides a compound of Formula I or Formula I-A in the manufacture of a medicament for decreasing blood flow in a subject.

In a further aspect, the present invention provides a compound of Formula I or Formula I-A or a pharmaceutical composition comprising a compound of Formula I or Formula I-A for use in a method of altering, preferably decreasing, motility in the gastrointestinal tract of a subject, e.g., a subject in need thereof.

In a further aspect, the present invention provides a method of altering, preferably decreasing, motility in the gastrointestinal tract of a subject in need thereof, the method comprising administering to said subject an effective amount of a compound of Formula I or Formula I-A or a pharmaceutical composition comprising a compound of Formula I or Formula I-A.

In a further aspect, the present invention provides a compound of Formula I or Formula I-A in the manufacture of a medicament for altering, preferably decreasing, motility in the gastrointestinal tract of a subject.

In a further aspect, the present invention provides a compound of Formula I or Formula I-A or a pharmaceutical composition comprising a compound of Formula I or Formula I-A for use in a method of treating a disorder of pituitary function, preferably wherein said disorder of pituitary function is a tumor, preferably prolactinomas, ACTH-secreting tumors, gonadotropin secreting tumors, TSH secreting tumors and non-secreting tumors, Rathke's cleft cysts, and craniopharyngiomas.

In a further aspect, the present invention provides a method of treating a disorder of pituitary function in a subject in need thereof, the method comprising administering to said subject an effective amount of a compound of Formula I or Formula I-A or a pharmaceutical composition comprising a compound of Formula I or Formula I-A preferably wherein said disorder of pituitary function is a tumor, preferably prolactinomas, ACTH-secreting tumors, gonadotropin secreting tumors, TSH secreting tumors and non-secreting tumors, Rathke's cleft cysts, and craniopharyngiomas.

In a further aspect, the present invention provides a compound of Formula I or Formula I-A in the manufacture of a medicament for treating a disorder of pituitary function, preferably wherein said disorder of pituitary function is a tumor, preferably prolactinomas, ACTH-secreting tumors, gonadotropin secreting tumors, TSH secreting tumors and non-secreting tumors, Rathke's cleft cysts, and craniopharyngiomas.

In one aspect, the present invention provides a compound of Formula I or Formula I-A or a pharmaceutical composition comprising a compound of Formula I or Formula I-A for use in a method of treating a metabolic disease, preferably wherein said metabolic disease is metabolic syndrome, obesity, preferably morbid obesity, or hypothalamic obesity. In some embodiments, said metabolic disease is a disorder of carbohydrate metabolism (e.g., type I diabetes mellitus, and/or type II diabetes mellitus), pre-diabetes, and/or hyperinsulinemia. In some embodiments, said metabolic disease is associated with the complications of diabetes mellitus, preferably wherein said disease associated with the complications of diabetes mellitus is nephropathy, retinopathy, angiopathy, and/or neuropathy.

In one aspect, the present invention provides a method of treating a metabolic disease in a subject in need thereof, the method comprising administering to said subject an effective amount of a compound of Formula I or Formula I-A or a pharmaceutical composition comprising a compound of Formula I or Formula I-A, preferably wherein said metabolic disease is metabolic syndrome, obesity, preferably morbid obesity, or hypothalamic obesity. In some embodiments, said metabolic disease is a disorder of carbohydrate metabolism (e.g., type I diabetes mellitus, and/or type II diabetes mellitus), pre-diabetes, and/or hyperinsulinemia. In some embodiments, said metabolic disease is associated with the complications of diabetes mellitus, preferably wherein said disease associated with the complications of diabetes mellitus is nephropathy, retinopathy, angiopathy, and/or neuropathy.

In one aspect, the present invention provides a compound of Formula I or Formula I-A in the manufacture of a medicament for treating a metabolic disease in a subject, preferably wherein said metabolic disease is metabolic syndrome, obesity, preferably morbid obesity, or hypothalamic obesity. In some embodiments, said metabolic disease is a disorder of carbohydrate metabolism (e.g., type I diabetes mellitus, and/or type II diabetes mellitus), pre-diabetes, and/or hyperinsulinemia. In some embodiments, said metabolic disease is associated with the complications of diabetes mellitus, preferably wherein said disease associated with the complications of diabetes mellitus is nephropathy, retinopathy, angiopathy, and/or neuropathy.

In one aspect, the present invention provides a compound of Formula I or Formula I-A or a pharmaceutical composition comprising a compound of Formula I or Formula I-A for use in a method of treating a disease associated with excess secretion in the gastrointestinal system, preferably wherein said disease associated with excess secretion of the gastrointestinal system is secretory diarrhea, infectious diarrhea, hormone-induced diarrhea due to increased secretion of gastrointestinal hormones (glucagon, carcinoid syndrome, VIPoma, glucagonoma, insulinoma), pancreatic and gut fistulas, abnormal gastrointestinal motility, and dumping syndrome.

In one aspect, the present invention provides a method of treating a disease associated with excess secretion of the gastrointestinal system in a subject in need thereof, the method comprising administering to said subject an effective amount of a compound of Formula I or Formula I-A or a pharmaceutical composition comprising a compound of Formula I or Formula I-A, preferably wherein said disease associated with excess secretion of the gastrointestinal system is secretory diarrhea, infectious diarrhea, hormone-induced diarrhea due to increased secretion of gastrointestinal hormones (glucagon, carcinoid syndrome, VIPoma, glucagonoma, insulinoma), pancreatic and gut fistulas, abnormal gastrointestinal motility, and dumping syndrome.

In one aspect, the present invention provides a compound of Formula I or Formula I-A in the manufacture of a medicament for treating diseases associated with excess secretion in the gastrointestinal system, preferably wherein said disease associated with excess secretion of the gastrointestinal system is secretory diarrhea, infectious diarrhea, hormone-induced diarrhea due to increased secretion of gastrointestinal hormones (glucagon, carcinoid syndrome, VIPoma, glucagonoma, insulinoma), pancreatic and gut fistulas, abnormal gastrointestinal motility, and dumping syndrome.

In one aspect, the present invention provides a compound of Formula I or Formula I-A or a pharmaceutical composition comprising a compound of Formula I or Formula I-A for use in a method of treating a disease of abnormal proliferation, preferably wherein said disease of abnormal proliferation is renal, hepatic and/or pancreatic cysts, or due to inflammation such as acute and chronic pancreatitis.

In one aspect, the present invention provides a method of treating a disease of abnormal proliferation in a subject in need thereof, the method comprising administering to said subject an effective amount of a compound of Formula I or Formula I-A or a pharmaceutical composition comprising a compound of Formula I or Formula I-A, preferably wherein said disease of abnormal proliferation is renal, hepatic and/or pancreatic cysts, or due to inflammation such as acute and chronic pancreatitis.

In one aspect, the present invention provides a compound of Formula I or Formula I-A in the manufacture of a medicament for treating diseases of abnormal proliferation, preferably wherein said disease of abnormal proliferation is renal, hepatic and/or pancreatic cysts, or due to inflammation such as acute and chronic pancreatitis.

In one aspect, the present invention provides a compound of Formula I or Formula I-A or a pharmaceutical composition comprising a compound of Formula I or Formula I-A for use in a method of treating a disease of abnormal bleeding or blood flow, preferably wherein said disease of abnormal bleeding or blood flow is gastrointestinal hemorrhage due to esophageal varices, ulcers, anastomoses, inflammatory bowel disease and/or cirrhosis.

In one aspect, the present invention provides a method of treating a disease of abnormal bleeding or blood flow in a subject in need thereof, the method comprising administering to said subject an effective amount of a compound of Formula I or Formula I-A or a pharmaceutical composition comprising a compound of Formula I or Formula I-A, preferably wherein said disease of abnormal bleeding or blood flow is gastrointestinal hemorrhage due to esophageal varices, ulcers, anastomoses, inflammatory bowel disease and/or cirrhosis.

In one aspect, the present invention provides a compound of Formula I or Formula I-A in the manufacture of a medicament for treating a disease of abnormal bleeding or blood flow, preferably wherein said disease of abnormal bleeding or blood flow is gastrointestinal hemorrhage due to esophageal varices, ulcers, anastomoses, inflammatory bowel disease and/or cirrhosis.

In one aspect, the present invention provides a compound of Formula I or Formula I-A or a pharmaceutical composition comprising a compound of Formula I or Formula I-A for use in a method of treating a disease or disorder in a subject in need thereof. In one aspect, the present invention provides a method of treating a disease or disorder in a subject in need thereof, the method comprising administering to said subject an effective amount of a compound of Formula I or Formula I-A or a pharmaceutical composition comprising a compound of Formula I or Formula I-A. In one aspect, the present invention provides a compound of Formula I or Formula I-A in the manufacture of a medicament for treating a disease or disorder. In some embodiments, said disease or disorder is selected from: acromegaly, pituitary gigantism, prolactinomas, ACTH-secreting tumors, gonadotropin secreting tumors, TSH secreting tumors and non-secreting tumors, Rathke's cleft cysts, craniopharyngiomas, metabolic syndrome, obesity, e.g., morbid obesity or hypothalamic obesity, type I and type II diabetes mellitus, pre-diabetes, hyperinsulinemia, nephropathy, retinopathy, angiopathy, neuropathy, secretory diarrhea, infectious diarrhea, hormone-induced diarrhea due to increased secretion of gastrointestinal hormones (glucagon, carcinoid syndrome, VIPoma, glucagonoma, insulinoma), pancreatic and gut fistulas, abnormal gastrointestinal motility, dumping syndrome, renal, hepatic and pancreatic cysts, acute and chronic pancreatitis, gastrointestinal hemorrhage due to esophageal varices, ulcers, anastomoses, inflammatory bowel disease, cirrhosis, neuroendocrine tumors of the pancreas, gastrointestinal tract, lung, thymus, breast, prostate, and adrenal glands, thyroid cancer, melanoma, meningioma, primary brain cancer, salivary gland cancer, ovarian cancer, and hepatocellular carcinoma.

In another aspect, the present invention provides for a method of diagnosis of a tumor, preferably a neuroendocrine tumor, preferably a carcinoid tumor, of a subject, comprising (a) administering a composition comprising the inventive compound to a subject, wherein preferably said composition further comprises a pharmaceutically or veterinary acceptable carrier; and (b) detecting said radionuclide. In a further very preferred embodiment, said diagnosis comprises in vivo Positron Emission Tomography (PET) imaging. In a preferred embodiment, said diagnosis comprises radionuclide imaging. In a further preferred embodiment, said tumor is selected from enteropancreatic neuroendocrine tumors, paragangliomas, pheochromocytomas, thyroid carcinomas and neuroblastomas. In another preferred embodiment, said tumor is a neuroendocrine tumor, preferably a carcinoid tumor, selected from (i) gastroenteropancreatic neuroendocrine tumors (GEP-NET); (ii) pulmonary and mediastinal neuroendocrine neoplasms (NEN); (iii) NEN of the adrenals or nervous system; (iv) meningiomas and low-grade gliomas; (v) solid tumors; and (vi) haematological malignancies. In a further very preferred embodiment, said tumor is a neuroendocrine tumor (NET), and wherein preferably said NET is selected from gastroenteropancreatic neuroendocrine tumors (GEP-NETs).

In a further aspect, the present invention provides for a method of visualizing malignant cells in a subject comprising administering to the subject an inventive compound of Formula I and further comprising a radionuclide or the inventive pharmaceutical composition, and visualizing said malignant cells by detecting the radionuclide.

In a further aspect, the present invention provides for a method of visualizing malignant cells having SSTR2 and SSTR5 in a subject comprising administering to the subject an inventive compound of Formula I and further comprising a radionuclide or the inventive pharmaceutical composition, and visualizing said malignant cells by detecting the radionuclide, wherein preferably said SSTR2 and SSTR5 bind to said compound.

In a further aspect, the present invention provides for a method of in vivo Positron Emission Tomography (PET) imaging of a tumor in a subject, wherein preferably said tumor is a neuroendocrine tumor (NET), and wherein further preferably said NET is selected from gastroenteropancreatic neuroendocrine tumors (GEP-NETs), comprising (a) administering a composition comprising the compound of Formula I in accordance with the present invention and comprising a radionuclide to a subject, wherein preferably said composition further comprises a pharmaceutically or veterinary acceptable carrier; and (b) detecting said radionuclide. In a further preferred embodiment, said tumor is selected from enteropancreatic neuroendocrine tumors, paragangliomas, pheochromocytomas, thyroid carcinomas and neuroblastomas. In another preferred embodiment, said tumor is a neuroendocrine tumor, preferably a carcinoid tumor, selected from (i) gastroenteropancreatic neuroendocrine tumors (GEP-NET); (ii) pulmonary and mediastinal neuroendocrine neoplasms (NEN); (iii) NEN of the adrenals or nervous system; (iv) meningiomas and low-grade gliomas; (v) solid tumors; and (vi) haematological malignancies. In a further preferred embodiment, said tumor, is a neuroendocrine tumor, preferably a carcinoid tumor. In another preferred embodiment, said tumor is a neuroendocrine tumor (NET), and wherein preferably said NET is selected from gastroenteropancreatic neuroendocrine tumors (GEP-NETs).

Therapeutically effective amounts of the inventive compounds should be administered under the guidance of a physician, and pharmaceutical compositions will usually contain the inventive compound in conjunction with a conventional, pharmaceutically or veterinary acceptable carrier. A therapeutically effective amount is considered to be a predetermined amount calculated to achieve the desired effect. The required dosage will vary with the particular treatment and with the duration of desired treatment.

Where the inventive compound is to be used for imaging or therapeutic treatments, poor shelf life of the radiolabeled compound and/or the short half-life of the radionuclide may require that the user carry out the labeling reaction with the radionuclide in the hospital or laboratory. In such instances, the various reaction ingredients may be provided to the user in the form of a kit. The manipulations necessary to perform the desired reaction should be as simple as possible to enable the user to prepare the radioactive labeled compound from the kit using facilities that normally be at one's disposal. Accordingly, a kit for preparing a radiopharmaceutical composition, for detecting and localizing or treating neuroendocrine, carcinoid or malignant tumors and their metastases in tissues might comprise (i) the inventive compound, an inert pharmaceutically acceptable carrier and/or formulating agent with optional adjuvants, (ii) a solution of a salt or chelate of a radioactive metal isotope, and (iii) instructions for use with a prescription for reacting the ingredients present in the kit.

Thus, in another aspect, the present invention provides for a kit for the diagnosis of a tumor, comprising: (a) the compound of Formula I in a suitable container, wherein said compound is either: (i) labeled with a radionuclide; (ii) unlabeled and provided with a radionuclide in a suitable container for labeling; or (iii) unlabeled and capable of being subsequently labeled with a radionuclide; and (b) instructions for use. In a preferred embodiment, said diagnosis comprises radionuclide imaging. In a further very preferred embodiment, said diagnosis comprises in vivo Positron Emission Tomography (PET) imaging.

In a further preferred embodiment, said tumor is selected from enteropancreatic neuroendocrine tumors, paragangliomas, pheochromocytomas, thyroid carcinomas and neuroblastomas. In another preferred embodiment, said tumor is a neuroendocrine tumor, preferably a carcinoid tumor, selected from (i) gastroenteropancreatic neuroendocrine tumors (GEP-NET); (ii) pulmonary and mediastinal neuroendocrine neoplasms (NEN); (iii) NEN of the adrenals or nervous system; (iv) meningiomas and low grade gliomas; (v) solid tumors; and (vi) haematological malignancies. In a further very preferred embodiment, said tumor is a neuroendocrine tumor (NET), and wherein preferably said NET is selected from gastroenteropancreatic neuroendocrine tumors (GEP-NETs).

In another aspect, the present invention provides for a kit for the treatment of a tumor, preferably a neuroendocrine tumor, further preferably a carcinoid tumor, overexpressing the somatostatin receptors, comprising: (a) the compound of Formula I in a suitable container, wherein, upon radionuclide labeling of said compound, the compound is present in a therapeutically effective amount for tumor treatment, and wherein said compound is either: (i) labeled with a radionuclide; (ii) unlabeled and provided with a radionuclide in a suitable container for labeling; or (iii) unlabeled and capable of being subsequently labeled with a radionuclide; and (b) instructions for use. In a further very preferred embodiment, said tumor overexpresses the somatostatin receptor subtypes 2 and/or 5.

Although the invention has been described with regard to its preferred embodiments, it should be understood that various changes and modifications as would be obvious to one having ordinary skill in the art may be made without departing from the scope of the invention that is set forth in the claims appended hereto. Although the claims variously define the invention in terms of the inventive compounds including a peptide sequence, it should be understood that such is intended to include nontoxic salts thereof that are well known to be the full equivalent thereof and that are most frequently administered.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

(2S)-3-(3-chloro-4-hydroxy-phenyl)-2-(9H-fluoren-9-ylmethoxycarbonylamino)propanoic acid was purchased commercially.

Unless otherwise stated, for the cAMP HTRF assay of Example 11 and the Radioligand Binding Competition assay of Example 12, on each day of experimentation and prior to the testing of compounds, reference compounds were tested at several concentrations in duplicate (n=2) to obtain a dose-response curve and an estimated EC₅₀ and/or IC₅₀ value. Reference values thus obtained were compared to historical values obtained from the same receptor and used to validate the experimental session. A session was only considered valid if the reference value was found to be within a 0.5 logs interval from the historical value. For replicate determinations, the maximum variability tolerated in the test was ±20% around the average of replicates.

Unless otherwise stated, HPLC was performed on a MZ Perfectsil™ column (5 μm C18 100 Å; 250×4.6 mm). Elution was performed using a gradient of solvent A (100% H₂O+0.1% TFA) and solvent B (80% ACN, 20% H₂O+0.1% TFA) at a flow rate of 1 mL/min using 30-55% solvent B. Compounds were detected at 220 nm.

### Materials

### Equipment Used

| Product | Source |
|---|---|
| Centrifuge ROTINA 380R | Hettich Lab Technology, |
| | Germany |
| Centrifuge MiniSpin Plus | Eppendorf, Germany |
| Revco Value PLUS −86° C. Freezer | Thermo scientific, USA |
| Freezer −20° C. | Liebherr, Germany |
| Duotronic Fridge +4° C. | Bosch, Germany |
| Laminar flow hood | Omicron, Italy |
| Functionline BB 16 CO₂ incubator | Heraeus, Germany |
| Optical microscope | CK Opympus Tokyo |
| DeNovix CellDrop | Diatech Labline, Italy |
| Mr. Frosty ™ Freezing Container | Thermo scientific, USA |
| Primo ® Pipette 0.5-10 μl | EuroClone, Italy |
| Primo ® Pipette 10-100 μl | EuroClone, Italy |
| Primo ® Pipette 100-1000 μl | EuroClone, Italy |
| Multichannel Pipette Research ® Plus | Eppendorf, Germany |
| Powerpette Pro | VWR, Italy |
| Vortex ZX3 | VELP scientific, Italy |
| Fluostar Omega Microplate Reader | BMG LABTECH, Germany |

### Consumable Materials

| Product | Source |
|---|---|
| Cell Culture Flasks 25 cm² | Greiner Bio-One, Germany |
| Cell Culture Flasks 75 cm² | EuroClone, Italy |
| Cell Culture Flasks 182 cm² | EuroClone, Italy |
| Nunc Cryotube vials | Thermo scientific, USA |
| Pipette Micro Tips 10 μl | BIOFIL, Italy |
| Pipette Micro Tips 200 μl | BIOFIL, Italy |
| Pipette Micro Tips 1000 μl | BIOFIL, Italy |
| Pipettes, serological 5 ml | BIOFIL, Italy |
| Pipettes, serological 10 ml | BIOFIL, Italy |
| Pipettes, serological 25 ml | BIOFIL, Italy |
| Reaction tubes 1.5 ml | Eppendorf, Germany |
| CLEARLine ® Centrifuge Tubes 50 ml | Biosigma, Italy |
| Centrifuge Tubes 15 ml | BIOFIL, Italy |
| Cell culture 24-wells plate | Corning Costar, USA |

### Chemicals and Reagents

| Product | Source |
|---|---|
| Dulbecco's Modified E Eagle's | Sigma-Aldrich, Germany |
| Medium (DMEM) | |
| Ham's F-12K (Kaighn's) Medium | Gibco, Germany |
| RPMI 1640 Medium | EuroClone, Italy |
| Dimethyl sulfoxide (DMSO) | Sigma-Aldrich, Germany |
| Fetal Bovine Serum (FBS) | Sigma-Aldrich, Germany |
| Horse Serum (HS) | Gibco, Germany |
| Phosphate Buffered Saline (PBS) | Sigma-Aldrich, Germany |
| Penicillin-Streptomycin (P/S) | EuroClone, Italy |
| L-glutamine | Sigma-Aldrich, Germany |
| Trypsin, 0.05% with EDTA | Sigma-Aldrich, Germany |
| Trypan Blue | Sigma-Aldrich, Germany |
| Collagen, Type IV, Human Placenta | Sigma-Aldrich, Germany |
| Epidermal Growth Factor (EGF) | Sigma-Aldrich, Germany |
| Fibroblast Growth Factor | Sigma-Aldrich, Germany |
| Mouse/Rat ACTH ELISA Kit | Abcam, UK |
| (adrenocorticotropic hormone) #ab263880 | |
| Rat/Mouse Growth hormone ELISA kit | Merck Millipore, USA |
| #EZRMGH-45K | |
| Human INS(Insulin) ELISA Kit #E-EL- | Elabscience, USA |
| H2665 | |

### Cell Culture

| Cell line | Species | Features | Medium |
|---|---|---|---|
| NT3 | Human | Lymph node derived cell line | RPMI 1640 |
| | | from a male patient with well- | +10% FBS |
| | | differentiated pNET. | +1% P/S |
| | | | +1% L-Glutamine |
| | | | (200 mM) |
| | | | +20 ng/ml EGF |
| | | | +10 ng/ml FGF |
| GH3 | Rat | Isolated from rat pituitary | F-12K |
| | | gland tumor. | +2.5% FBS |
| | | | +15% HS |
| | | | +1% P/S |
| | | | +1% L-Glutamine |
| | | | (200 mM) |
| AtT-20 | Mouse | Isolated from mouse anterior | DMEM |
| | | pituitary gland tumor. | +10% FBS |
| | | | +1% P/S |
| | | | +1% L-Glutamine |
| | | | (200 mM) |

### Sub-Culturing

Unless otherwise noted, cells were cultured in the respective medium and incubated at 37° C. in an atmosphere containing 5% CO₂. All cell lines were cultivated under the same experimental conditions. For the sub-culturing, cells were washed with PBS and incubated with 0.05% trypsin/EDTA for 3-5 min at 37° C. Medium with FBS was given to inhibit trypsin. Cells were split by a ratio between 1:2 and 1:10 depending on the cell line and the confluence of the cells. Cells were used for a maximum of 10 passages after thawing. For NT3 cells the cell culture flasks were previously coated with Collagen (Type IV, Human Placenta) (50 μg/ml in PBS) before the seeding.

### Cryopreservation of Cells

Unless otherwise noted, cell lines were cryopreserved at early passages. To this purpose, cells in the log growth phase were pelleted by centrifugation for 5 minutes at 13,400 rpm. The supernatant was discarded, and the cell pellet was resuspended in culture medium containing 10% DMSO. The cell suspension was aliquoted in cryovials, placed in a cell freezing container and stored at −80° C. Long-term storage was carried out in liquid nitrogen.

To thaw cells, cryovials were thawed in a 37° C. water bath, and the cell suspension was given into complete culture medium immediately after thawing. After centrifugation at 13,400 rpm for 5 minutes, the cells were resuspended in culture medium and placed in a cell culture flask.

### Example 1

### Synthesis of (2S)-2-(9H-fluoren-9-ylmethoxycarbonylamino)-3-(4-hydroxy-3-iodo-phenyl) propanoic acid) ((2S)-Fmoc-(3-I)-Tyr-OH; Compound P1)

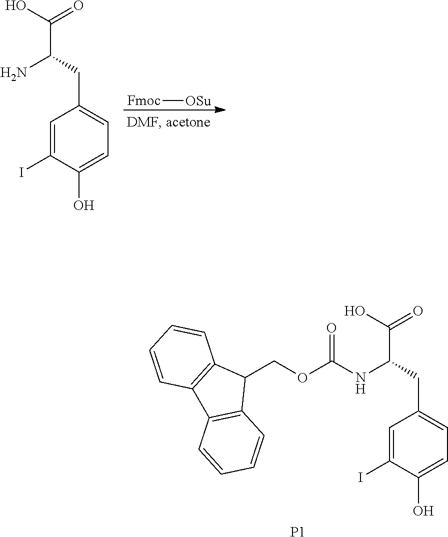

Commercially available 3-iodo-L-tyrosine (Chemimpex) (1.00 g, 3.256 mmol) was suspended in a mixture of N,N-dimethylformamide and acetone (10 mL), and the suspension was cooled to 0° C. After 10 min stirring, 10% sodium bicarbonate solution (7 mL, 8.141 mmol) was added dropwise, followed by 9-fluorenylmethyl N-succinimidyl carbonate (1.318 g, 3.908 mmol) and N,N-dimethylformamide (25 mL). The reaction mixture was stirred at room temperature for 2 h. The mixture was concentrated and to the residue was added water (25 mL) and washed with ethyl acetate (1×5 mL). The aqueous layer was cooled to 0° C. and acidified to pH 1 with 10% potossium hydrogen sulphate (11.5 mL). The mixture was extracted with ethyl acetate (15 mL then 4×5 mL). The combined organic layers were washed with brine (2×10 mL), dried over magnesium sulphate, filtered and evaporated. The crude product was dried under vacuum overnight. Chloroform (30 mL) was added to the crude product and the white precipitate was filtered to give the title compound (1.13 g, 2.135 mmol, 66%) as a white solid. LCMS: 97%, t_{R}=2.031 min, m/z=528.0 [M−H]⁻.

### Example 2

### Synthesis of ((2R)-3-(4-amino-3-iodo-phenyl)-2-(tert-butoxycarbonylamino)propanoic acid) (Boc-(4-amino-3-iodo)-D-Phe-OH; Compound P2)

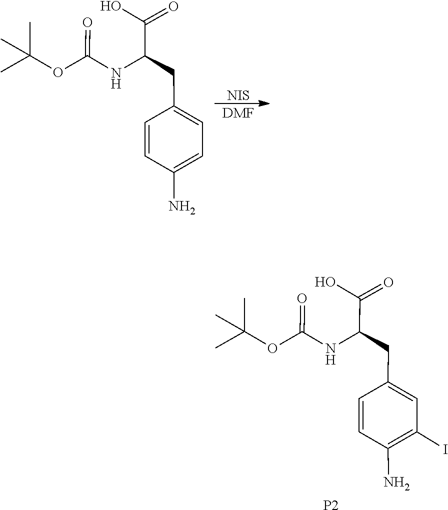

To a solution of Boc-(4-amino)-D-Phe-OH (CombiBlocks) (3.50 g, 12.485 mmol) in N,N-dimethylformamide (189 mL) was added N-iodosuccinimide (3.09 g, 13.734 mmol) and the mixture was stirred at room temperature for 1.5 h. The reaction mixture was diluted with water (200 mL) and extracted with ethyl acetate (3×100 mL). The combined organic layers were washed with brine (1×50 mL), dried over magnesium sulphate, filtered, and evaporated. The crude product was purified by preparative HPLC to give 2.14 g off-white title compound in two batches. LCMS: 100%, t_{R}=1.768 min, m/z=404.9 [M−H]⁻.

### Example 3

### Synthesis of ((2R)-3-(4-amino-3-chloro-phenyl)-2-(tert-butoxycarbonylamino)propanoic acid) (Boc-(4-amino-3-chloro)-D-Phe-OH; Compound P3)

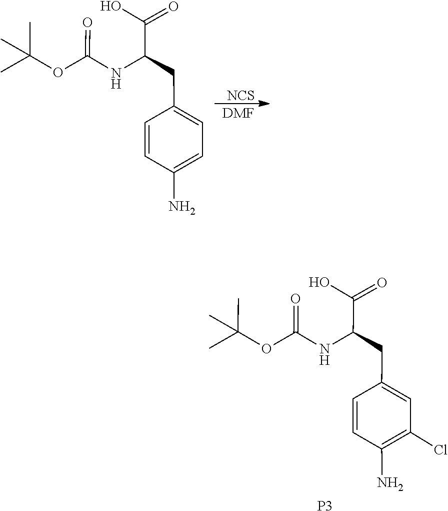

To a solution of Boc-(4-amino)-D-Phe-OH (2.00 g, 7.135 mmol) in N,N-dimethylformamide (108 mL) was added N-chlorosuccinimide (1.05 g, 7.848 mmol) and the mixture was stirred at room temperature for 2 h. The reaction mixture was evaporated and the residue was purified by preparative HPLC to give the title compound (0.89 g, 2.828 mmol, 40%) as an off white solid. LCMS: 100%, t_{R}=1.687 min, m/z=313.0 [M−H]⁻.

### Example 4

### Synthesis of (2R)-3-(4-amino-3-bromo-phenyl)-2-(tert-butoxycarbonylamino)propanoic acid

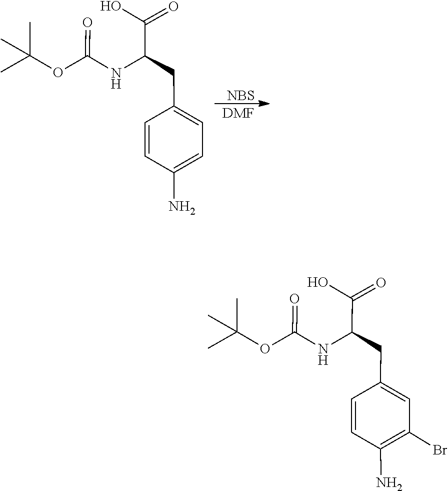

To a solution of (2R)-3-(4-aminophenyl)-2-(tert-butoxycarbonylamino)propanoic acid (Combi-Blocks) (3.00 g, 10.702 mmol) in N,N-dimethylformamide (150 mL) was added N-bromosuccinimide (2.10 g, 11.804 mmol) and the mixture was stirred at room temperature for 1 d. The reaction mixture was evaporated and the residue was purified by preparative HPLC to give the title compound (3.06 g, 8.518 mmol, 80%) as an off white powder. LCMS: 97%, t_{R}=1.285 min, m/z=358.9 [M−H]⁻.

### Example 5

### Synthesis of (2R)-3-(4-amino-3-fluoro-phenyl)-2-(tert-butoxycarbonylamino)propanoic acid

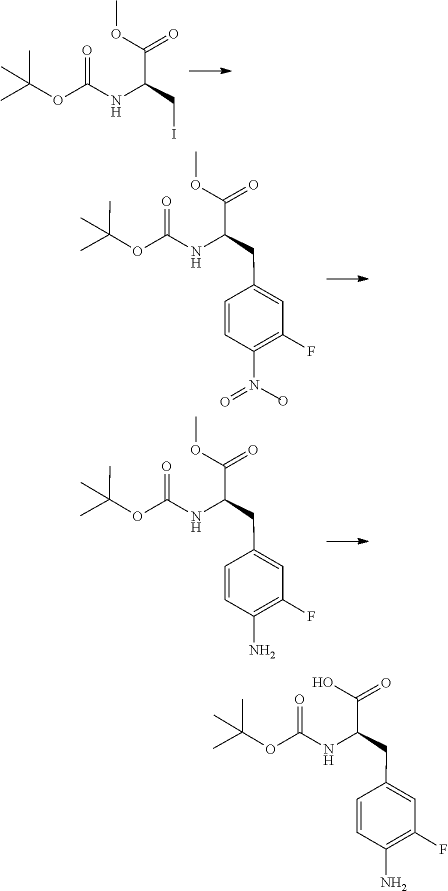

### Synthesis of methyl (2R)-2-(tert-butoxycarbonylamino)-3-(3-fluoro-4-nitro-phenyl)propanoate

To a suspension of zinc (0.177 g, 2.704 mmol) in anhydrous N,N-dimethylformamide (3.8 mL) was added iodine (0.039 g, 0.152 mmol) at room temperature. The mixture was stirred at room temperature for 20 min while the color changed from brown to grey. To the reaction mixture was added methyl (2S)-2-(tert-butoxycarbonylamino)-3-iodo-propanoate (Accelachem) (0.50 g, 1.519 mmol) along with iodine (0.039 g, 0.152 mmol). The stirring was continued for 2 h at room temperature. To this mixture was added a solution of 4-bromo-2-fluoro-1-nitro-benzene (0.334 g, 1.519 mmol), 2-dicyclohexyphosphino-2′,6′-dimethoxybiphenyl (0.037 g, 0.091 mmol) and tris(dibenzylideneacetone)dipalladium(0) (0.042 g, 0.046 mmol) in anhydrous N,N-dimethylformamide (7.5 mL). The reaction mixture was stirred at 65° C. for 17 h. The reaction was repeated in three more batches. The four batches were combined and evaporated. The residue was dissolved in a mixture of ethyl acetate (20 mL) and water (20 mL). The layers were separated, the aqueous layer was extracted with ethyl acetate (3×20 mL). The combined organic layers were washed with brine (1×20 mL), dried over magnesium sulphate, filtered and evaporated. The residue was purified by gradient silica gel column chromatography eluting with n-heptane:ethyl acetate (10:1-43:1) to give the title compound (0.80 g, 2.339 mmol, 22%) as a brown powder. LCMS: 94%, t_{R}=1.561 min, m/z=243.1 [M+H-tBu]⁺.

### Synthesis of methyl (2R)-3-(4-amino-3-fluoro-phenyl)-2-(tert-butoxycarbonylamino) propanoate

To a solution of methyl (2R)-2-(tert-butoxycarbonylamino)-3-(3-fluoro-4-nitro-phenyl)propanoate (0.735 g, 2.147 mmol) in methanol (7.5 mL) was added 10% palladium on carbon (228 mg). The reaction mixture was stirred at room temperature for 2 h under hydrogen. The mixture was filtered through a pad of Celite and the Celite was washed with methanol (4×5 mL). The combined filtrates were evaporated to give the title compound (0.643 g, 2.059 mmol, 96) as a dark brown oil. LCMS: 87%, t_{R}=1.328 min, m/z=213.2 [M+H-tBu]⁺.

### Synthesis of (2R)-3-(4-amino-3-fluoro-phenyl)-2-(tert-butoxycarbonylamino)propanoic acid

Methyl (2R)-3-(4-amino-3-fluoro-phenyl)-2-(tert-butoxycarbonylamino)propanoate (2.0 g, 6.403 mmol) was suspendedn in a mixture of tetrahydrofuran (20 mL) and water (20 mL). To the stirred mixture was added lithium hydroxide monohydrate (0.806 g, 19.210 mmol) and the reaction mixture was stirred at room temperature for 30 min. The mixture was concentrated and diluted with water (30 mL) and diethyl ether (30 mL). The layers were separated and the aqueous layer was washed with diethyl ether (1×30 mL). The aqueous layer was cooled to 0° C. and acidified to pH 3 with 10% aqueous potassium hydrogen sulphate solution then extracted with ethyl acetate (3×20 mL). The combined organic layers were dried over magnesium sulphate, filtered and evaporated to give the title compound (1.42 g, 4.760 mmol, 74%) as an off-white foam. LCMS: 97%, t_{R}=1.056 min, m/z=199.1 [M+H-tBu]⁺.

### Example 6

### Synthesis of (2S)-2-(9H-fluoren-9-ylmethoxycarbonylamino)-3-(4-hydroxy-3-bromo-phenyl)propanoic acid

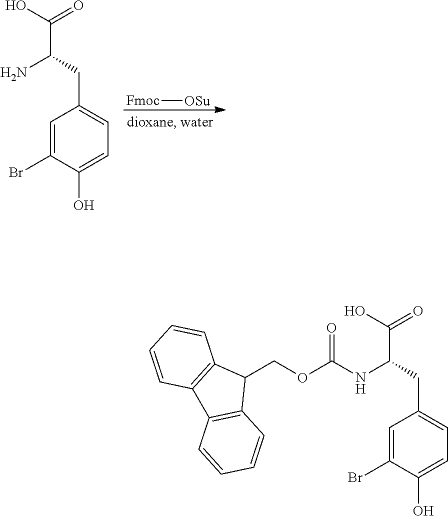

(2S)-2-Amino-3-(3-bromo-4-hydroxy-phenyl)propanoic acid (Enamine) (0.50 g, 1.922 mmol) was suspended in a mixture of 1,4-dioxane (10 mL) and water (10 mL) and the suspension was cooled to 0° C. After 10 min stirring, sodium bicarbonate (0.388 g, 4.14 mmol) was added, followed by 9-fluorenylmethyl N-succinimidyl carbonate (0.713 g, 2.115 mmol). The reaction mixture was stirred at room temperature for 2 h and diluted with ethyl acetate (10 mL). The layers were separated. The aqueous layer was cooled to 0° C. and acidified to pH 1 with 10% aqueous potassium hydrogen sulphate solution. The mixture was extracted with ethyl acetate (3×20 mL). The combined organic layers were washed with brine (2×10 mL), dried over magnesium sulphate, filtered and evaporated. The crude product was purified by preparative HPLC to give the title compound (0.60 g, 1.244 mmol, 65%) as a brown oil. LCMS: 97%, t_{R}=1.617 min, m/z=481.9 [M−H]⁻.

### Example 7

### Synthesis of (2S)-2-(9H-fluoren-9-ylmethoxycarbonylamino)-3-(4-hydroxy-3-fluoro-phenyl)propanoic acid

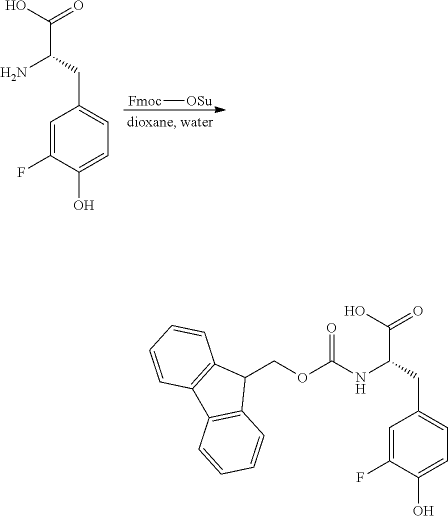

(2S)-2-Amino-3-(3-fluoro-4-hydroxy-phenyl)propanoic acid (Ambeed) (1.50 g, 7.531 mmol) was suspended in a mixture of 1,4-dioxane (40 mL) and water (40 mL) and the suspension was cooled to 0° C. After 10 min stirring, sodium bicarbonate (1.581 g, 18.827 mmol) was added, followed by 9-fluorenylmethyl N-succinimidyl carbonate (3.048 g, 9.037 mmol). The reaction mixture was stirred at room temperature for 2 h and diluted with diethyl ether (10 mL). The layers were separated. The aqueous layer was cooled to 0° C. and acidified to pH 1 with 10% aqueous potassium hydrogen sulphate solution. The mixture was extracted with ethyl acetate (3×20 mL). The combined organic layers were washed with brine (2×10 mL), dried over magnesium sulphate, filtered and evaporated to give the title compound (3.00 g, 7.119 mmol, 94%) as an off-white solid. LCMS: 93%, t_{R}=1.517 min, m/z=420.0 [M−H]⁻.

### Example 8

### Synthesis of (2R)-3-(4-amino-3-chloro-phenyl)-2-(9H-fluoren-9-ylmethoxycarbonylamino)propanoic acid

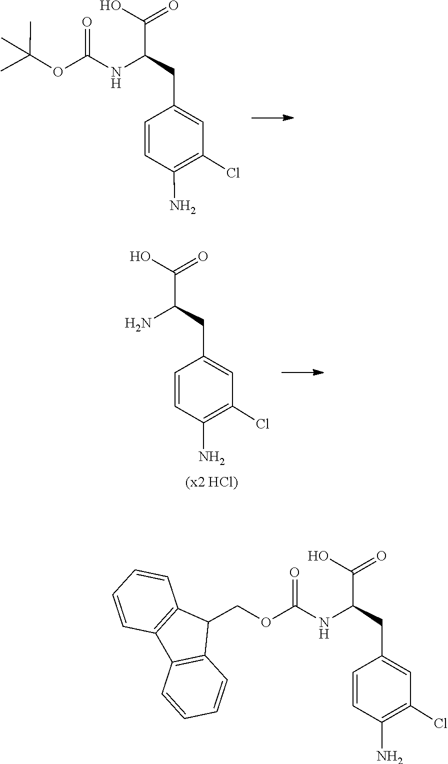 [SMILES: CC(C)(C)OC(=O)N[C@H](Cc1ccc(N)c(Cl)c1)C(=O)O ; Nc1ccc(C[C@@H](N)C(=O)O)cc1Cl ; Nc1ccc(C[C@@H](NC(=O)OCC2c3ccccc3-c3ccccc32)C(=O)O)cc1Cl]

### Synthesis of (2R)-2-amino-3-(4-amino-3-chloro-phenyl)propanoic acid dihydrochloride

To a solution of (2R)-3-(4-amino-3-chloro-phenyl)-2-(tert-butoxycarbonylamino)propanoic acid (2.0 g, 5.568 mmol) in 1,4-dioxane (10 mL) was added 7.06M hydrochloric acid in 1,4-dioxane (15.7 mL, 111.35 mmol). The reaction mixture was stirred at room temperature for 16 h and evaporated to give the title compound (2.12 g, 7.372 mmol, crude) as a light brown crystalline solid. LCMS: 96%, t_{R}=0.193 min, m/z=215.1 [M+H]⁺.

### Synthesis of (2R)-3-(4-amino-3-chloro-phenyl)-2-(9H-fluoren-9-ylmethoxycarbonylamino)propanoic acid

(2R)-2-Amino-3-(4-amino-3-chloro-phenyl)propanoic acid dihydrochloride (2.11 g, 7.337 mmol, crude) was dissolved in 9% aqueous sodium carbonate solution (16.8 mL) and the suspension was cooled to 0° C. To the reaction mixture was added a mixture of 9-fluorenylmethyl N-succinimidyl carbonate (2.475 g, 7.337 mmol) and 1,4 dioxane (24 mL). The reaction mixture was allowed to warm to room temperature and stirred for 1 h. To the reaction mixture was added 10% aqueous citric acid solution to adjust pH to 5-6. The layers were separated, the aqueous layer was extracted with chloroform (4×40 mL), followed by a mixture of chloroform:2-propanol (3:1, 2×20 mL). The combined organic layers were dried over magnesium sulphate, filtered and evaporated. The crude product was purified by preparative HPLC to give the title compound (1.434 g, 3.282 mmol, 59% for two steps) as an off-white crystalline solid. LCMS: 100%, t_{R}=1.643 min, m/z=437.1 [M+H]⁺.

### Example 9

### Synthesis of (2R)-3-(4-amino-3-bromo-phenyl)-2-(9H-fluoren-9-ylmethoxycarbonylamino)propanoic acid

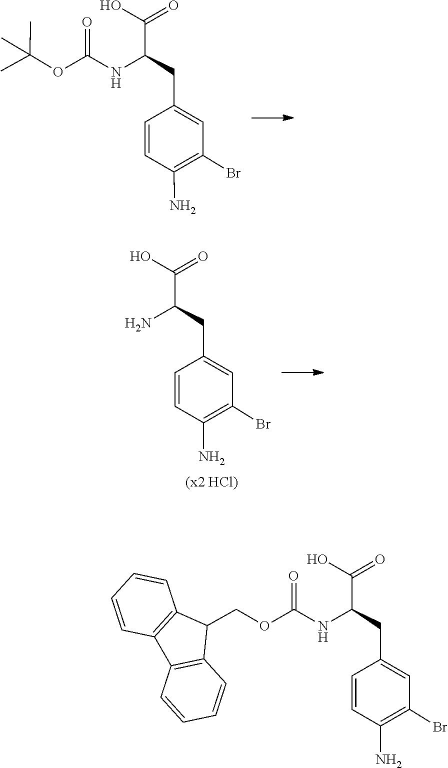 [SMILES: CC(C)(C)OC(=O)N[C@H](Cc1ccc(N)c(Br)c1)C(=O)O ; Nc1ccc(C[C@@H](N)C(=O)O)cc1Br ; Nc1ccc(C[C@@H](NC(=O)OCC2c3ccccc3-c3ccccc32)C(=O)O)cc1Br]

### Synthesis of (2R)-2-amino-3-(4-amino-3-bromo-phenyl)propanoic acid dihydrochloride

To a solution of (2R)-3-(4-amino-3-bromo-phenyl)-2-(tert-butoxycarbonylamino)propanoic acid (1.44 g, 4.009 mmol) in 1,4-dioxane (6 mL) was added 7.06M hydrochloric acid in 1,4-dioxane (11.3 mL, 80.17 mmol). The reaction mixture was stirred at room temperature for 2 h and evaporated to give the title compound (1.66 g, 4.999 mmol, crude) as an off-white crystalline solid. LCMS: 100%, t_{R}=0.214 min, m/z=261.1 [M+H]⁺.

### Synthesis of (2R)-3-(4-amino-3-bromo-phenyl)-2-(9H-fluoren-9-ylmethoxycarbonylamino)propanoic acid

(2R)-2-Amino-3-(4-amino-3-bromo-phenyl)propanoic acid dihydrochloride (1.55 g, 4.668 mmol, crude) was dissolved in 9% aqueous sodium carbonate solution (10.7 mL) and the suspension was cooled to 0° C. To the reaction mixture was added a mixture of 9-fluorenylmethyl N-succinimidyl carbonate (1.575 g, 4.668 mmol) and 1,4 dioxane (15.2 mL). The reaction mixture was allowed to warm to room temperature and stirred for 1 h. The reaction mixture was combined with a batch, which was initiated from 100 mg of dihydrochloride. To the reaction mixture was added 10% aqueous citric acid solution to adjust pH to 5-6. The layers were separated, the aqueous layer was extracted with chloroform (4×40 mL), followed by a mixture of chloroform:2-propanol (3:1, 2×20 mL). The combined organic layers were dried over magnesium sulphate, filtered and evaporated. The crude product was purified by preparative HPLC to give the title compound (1.447 g, 3.005 mmol, 75% for two steps) as a white crystalline solid. LCMS: 100%, t_{R}=1.662 min, m/z=483.1 [M+H]⁺.

### Example 10

### Synthesis of Cyclic Peptide Analogs

Peptides were synthesized manually using an Fmoc-Rink-Amide MBHA resin, 0.71 mmol/g, 0.33 mmol, 0.464 g of resin. Cyclization was allowed to proceed for 24 h in water in the presence of potassium hexacyanoferrate (III).

(4-amino-3-I)-D-Phe-c[Cys-(3-Cl)-Tyr-D-Trp-Lys-Val-Cys]-Thr-NH₂ (Compound 1): Cyclization provided 305 mg crude product of which 14.5 mg of pure peptide was obtained in 4.75% overall yield. Purification was achieved by RP HPLC. Purity and identity of the peptide was established by analytical HPLC and LCMS, respectively: ESI-MS (m/z): [MH]⁺, C₅₀H₆₆ClIN₁₂O₁₀S₂; expected: 1221.62; found: 1221.21.

(4-amino-3-Cl)-D-Phe-c[Cys-(3-I)-Tyr-D-Trp-Lys-Val-Cys]-Thr-NH₂ (Compound 2): Cyclization provided 293 mg crude product of which 13.76 mg of pure peptide was obtained in 4.7% overall yield. Purification was achieved by RP HPLC. Purity and identity of the peptide was established by analytical HPLC and LCMS, respectively: ESI-MS (m/z): [MH]⁺, C₅₀H₆₆ClIN₁₂O₁₀S₂; expected: 1221.62; found: 1221.26.

(4-amino-3-Cl)-D-Phe-c[Cys-(3-Cl)-Tyr-D-Trp-Lys-Val-Cys]-Thr-NH₂ (Compound 3): Cyclization provided 313 mg crude product of which 19.1 mg of pure peptide was obtained in 6.1% overall yield. Purification was achieved by RP HPLC. Purity and identity of the peptide was established by analytical HPLC and LCMS, respectively: ESI-MS (m/z): [MH]⁺, C₅₀H₆₆Cl₂N₁₂O₁₀S₂; expected: 1130.16; found: 1131.21.

(4-amino-3-F)-D-Phe-c[Cys-(3-F)-Tyr-D-Trp-Lys-Val-Cys]-Thr-NH₂ (Compound 4) was synthesized manually, as described in the general experimental procedures, using a Fmoc-Rink-Amide MBHA resin, 0.71 mmol/g, 0.5 mmol, 0.705 g of resin. Cyclization was allowed to proceed for 24 h in water in the presence of potassium hexacyanoferrate(III). The crude yield was 285 mg, out of which 9.45 mg of pure peptide was obtained, 3.15% overall. Purification was achieved with 10% solvent B over 10 min followed by 40% solvent B over 70 min (4 mL/min, λ=220 nm), and finally isocratic elution. The purity of the compound was ascertained by analytical HPLC using a gradient of 35% solvent B, 0 min, and isocratic elution with 50% solvent B, 15 min. ESI-MS (m/z): [MH]⁺ C₅₀H₆₇F₂N₁₂O₁₀S₂ expected 1098.32, found 1099.18.

(4-amino-3-Br)-D-Phe-c[Cys-(3-Br)-Tyr-D-Trp-Lys-Val-Cys]-Thr-NH₂ (Compound 5) was synthesized manually, as described in the general experimental procedures, using a Fmoc-Rink-Amide MBHA resin, 0.71 mmol/g, 0.5 mmol, 0.705 g of resin. Cyclization was allowed to proceed for 24 h in water in the presence of potassium hexacyanoferrate(III). The crude yield was 262 mg, out of which 6.3 mg of pure peptide were obtained, 2.40% overall. Purification was achieved with 15% solvent B over 15 min followed by 45% solvent B over 75 min (4 mL/min, λ=220 nm), and finally isocratic elution. The purity of the compound was ascertained by analytical HPLC using a gradient of 40% solvent B, 0 min, and isocratic elution with 55% solvent B, 15 min. ESI-MS (m/z): [MH]⁺ C₅₀H₆₇Br₂N₁₂O₁₀S₂ expected 1221.06, found 1222.01.

### Example 11

cAMP HTRF Assay

CHO-K1 cells (accession number NP_001041.1) expressing human recombinant receptor SST2 were grown prior to the test in media without antibiotic and were detached by gentle flushing with PBS-EDTA (5 mM EDTA). The cells were recovered by centrifugation and resuspended in assay buffer (KRH: 5 mM KCl, 1.25 mM MgSO₄, 124 mM NaCl, 25 mM HEPES, 13.3 mM Glucose, 1.25 mM KH₂PO₄, 1.45 mM CaCl₂, 0.5 g/l BSA, supplemented with 1 mM IBMX).

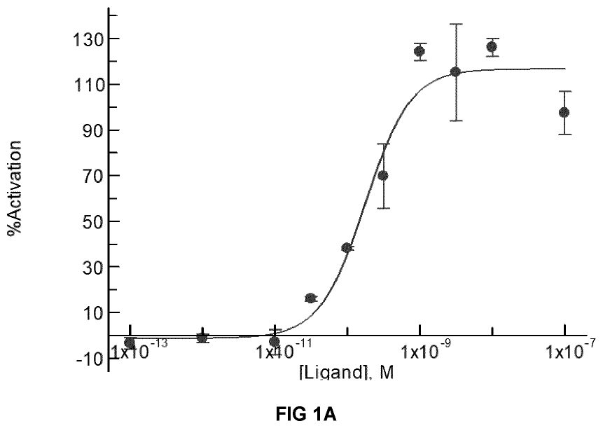

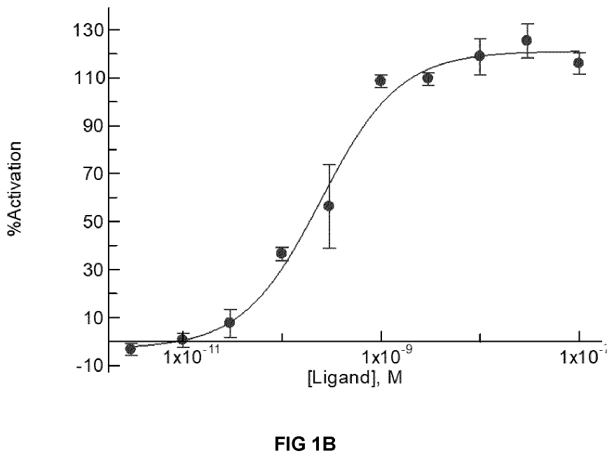

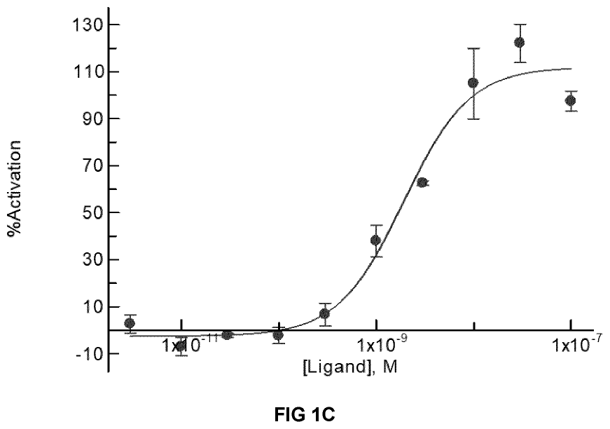

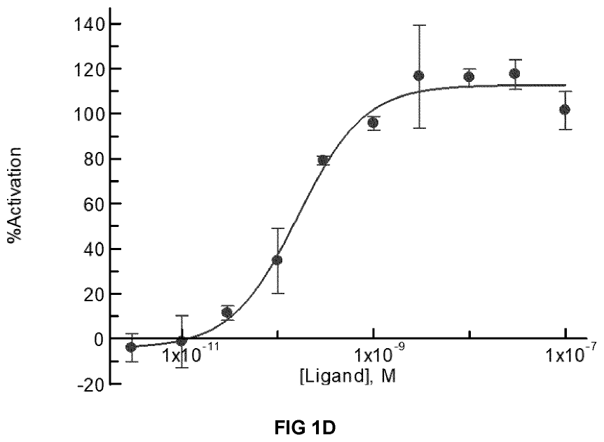

Dose-response curves for the inventive compounds were generated in parallel with the reference compound SS-28 (together, the “test compounds”). In a 96-well plate, 12 μl of cells were mixed with 6 μl of the test compound at increasing concentrations and 6 μl of forskolin, then incubated for 30 min at room temperature. The final concentrations of test compound were 0.003 nM; 0.01 nM; 0.03 nM; 0.1 nM; 0.3 nM; 1 nM; 3 nM; 10 nM; 30 nM; and 100 nM. After addition of lysis buffer containing cAMP-d2 and anti-cAMP cryptate detection reagents, the plates were incubated for one hour at room temperature, and fluorescence ratios were measured using an HTRF kit according to the manufacturer specification. The results are shown below in Table 1 and in FIGS. 1A-1D.

TABLE 1
SST2 EC₅₀ Values for Compounds 3, 4
and 5 as Measured by cAMP HTRF Assay

| Compound ID | EC₅₀ (nM) |
|---|---|
| SS-28 | 0.18 |
| Compound 3 | 0.25 |
| Compound 4 | 1.91 |
| Compound 5 | 0.16 |

### Example 12

### Radioligand Binding Competition Assay

Competition binding was performed in duplicate in the wells of a 96-well Master Block® plate (Greiner, 786201) containing binding buffer, membrane extracts from CHO cells, radiotracer (^{[125I]}SS-14) and test compound (i.e., the inventive compounds or SS-28 as a reference). For the SST2 assay, membrane extracts from CHO-K1 cells (accession number NP_001041.1) were used. For the SST5 assay, membrane extracts from CHO-K1 cells (accession number NP_001044.1) were used.

Nonspecific binding was determined by co-incubation with 200-fold excess of the cold competitor ^{[125I]}SS-14. The samples were incubated at a final volume of 0.1 mL at a temperature and for a duration optimized for each receptor and then filtered over filter plates. Filters were washed six times with 0.5 mL of ice-cold washing buffer (optimized for each receptor) and 50 μL of Microscint™ 20 (Packard) were added in each well. The plates were incubated 15 minutes on an orbital shaker and then counted with a TopCount™ for 1 min/well. Experiments were performed on different days, and a comparison to SS-28 was made for each experiment (Day 1: Compounds 1 and 2; Day 2: Compound 3; Day 3: Compounds 4 and 5).

Dose-response data from test compounds were analyzed with XLfit (IDBS) software (XL Fit Model 203 4 Parameter Logistic Model) using nonlinear regression applied to a sigmoidal dose-response model and Equations 1-3, below:
A: Bottom
B: Top
C: Log EC₅₀
D: Hill

$$fit = ( A + ( ( B - A ) / ( 1 + ( ( ( 10 \hat{} C ) / x ) \hat{} D ) ) ) ) \qquad Equation 1$$

$$inv = ( ( 10 \hat{} C ) / ( ( ( ( B - A ) / ( y - A ) ) - 1 ) \hat{} ( 1 / D ) ) ) \qquad Equation 2$$

$$res = ( y - fit ) \qquad Equation 3$$

Results are shown below in Tables 2-7 and FIGS. 2A-2H and 3A-3H.

TABLE 2
SST2 IC₅₀ values for Compounds 1 and 2 as Measured
by Radioligand Binding Competition Assay

| Compound ID | IC₅₀ (nM) |
|---|---|
| SS-28 | 0.21 |
| Compound 1 | 0.37 |
| Compound 2 | 0.74 |

TABLE 3
SST2 IC₅₀ value for Compound 3 as Measured
by Radioligand Binding Competition Assay

| Compound ID | IC₅₀ (nM) |
|---|---|
| SS-28 | 0.17 |
| Compound 3 | 0.48 |

TABLE 4
SST2 IC₅₀ values for Compounds 4 and 5 as Measured
by Radioligand Binding Competition Assay

| Compound ID | IC₅₀ (nM) |
|---|---|
| SS-28 | 0.10 |
| Compound 4 | 2.60 |
| Compound 5 | 0.54 |

TABLE 5
SST5 IC₅₀ values for Compounds 1 and 2 as Measured
by Radioligand Binding Competition Assay

| Compound ID | IC₅₀ (nM) |
|---|---|
| SS-28 | 0.076 |
| Compound 1 | 1.22 |
| Compound 2 | 0.73 |

TABLE 6
SST5 IC₅₀ values for Compound 3 as Measured
by Radioligand Binding Competition Assay

| Compound ID | IC₅₀ (nM) |
|---|---|
| SS-28 | 0.31 |
| Compound 3 | 0.86 |

TABLE 7
SST5 IC₅₀ values for Compounds 4 and 5 as Measured
by Radioligand Binding Competition Assay

| Compound ID | IC₅₀ (nM) |
|---|---|
| SS-28 | 0.17 |
| Compound 4 | 3.41 |
| Compound 5 | 0.79 |

### Example 13

Inhibition of Hormone Release from Mouse Pituitary Corticotrope Adenoma (AtT-20) Cells

Corticotropes are the cells of the anterior pituitary that secrete the hormone adrenocorticotropin (ACTH). Tumors of the corticotrope cells cause over-secretion of ACTH, which stimulates the adrenal glands to produce high levels of cortisol; this pituitary disease is called Cushing's disease. The mouse AtT-20 cell line is the standard model to study ACTH secretion by pituitary corticotrope tumors.

AtT-20 cells were seeded in 24-well plates (20,000 cells/well) in complete culture medium and incubated overnight at standard incubator conditions (37° C., 5% CO₂) to promote adhesion. Then the supernatant was discarded, and the cells were incubated with the following compounds:
Compound 1;
Compound 2;
Compound 3;
Compound 4;
Compound 5.

All compounds were studied at concentrations of 5 nM and 10 nM; untreated cell controls were also studied. A biological duplicate was reproduced for each concentration of each drug. The treatment exposure period was 72 h. At the 72 h time-point, cell supernatant was collected and deposited into 1.5 mL test tubes that were then centrifuged at 13,400 rpm for 5 minutes to remove any cell residues. The samples were stored at −80° C. until the time of analysis.

ACTH levels were quantified using a Sandwich-ELISA technique ((Abcam Mouse/Rat ACTH ELISA Kit #ab263880) according to the manufacturer's instructions. Samples and standards were added individually to the wells, followed by the antibody mix. After incubation, the wells were washed to remove unbound material, and development solution was added, which was catalysed, and the reaction was subsequently stopped. The signal was generated proportionally to the amount of bound analyte and the intensity was measured at 450 nm. Results are shown in Table 8 and in FIG. 4A-4E.

TABLE 8
Change in ACTH Secretion by AtT-20 Cells Following
Treatment for 72 hours with Compounds 1-5

| | Fold Change in |
|---|---|
| Compound ID | Measured ACTH* |
| Control (no treatment) | 1.00 |
| Compound 1; 5 nM | 0.36 |
| Compound 1; 10 nM | 0.47 |
| Compound 2; 5 nM | 0.62 |
| Compound 2; 10 nM | 0.89 |
| Compound 3; 5 nM | 0.68 |
| Compound 3; 10 nM | 0.76 |
| Compound 4; 5 nM | 0.37 |
| Compound 4; 10 nM | 0.37 |
| Compound 5; 5 nM | 0.68 |
| Compound 5; 10 nM | 0.77 |

*In comparison to untreated (control) AtT-20 cells

### Example 14

Inhibition of Hormone Release from Rat Pituitary Somatotrope (GH3) Cells

The somatotrope cells of the anterior pituitary produce growth hormone (GH). Pituitary tumors of these cells are termed somatotropinomas and lead to excess GH secretion causing either pituitary gigantism in children and adolescents, or acromegaly in adults. GH3 cells are derived from rat somatotrope tumors and are the standard model used for the study of somatotropinomas and GH secretion. GH3 cells express multiple SSTRs, making them an appropriate model to study somatostatin analog characteristics.

GH3 cells were seeded in 24-well plates at a density of 100,000 cells/well, in serum-free culture medium supplemented with the following drugs:
Compound 1;
Compound 2;
Compound 3;
Compound 4;
Compound 5.

All compounds were studied at concentrations of 5 nM and 10 nM; untreated cell controls were also studied. A biological duplicate was reproduced for each concentration of each drug. The treatment exposure period was 72 h. At the 72 h time-point, cell supernatant was collected and deposited into 1.5 mL test tubes that were then centrifuged at 13,400 rpm for 5 minutes to remove any cell residues. The samples were stored at −80° C. until the time of analysis.

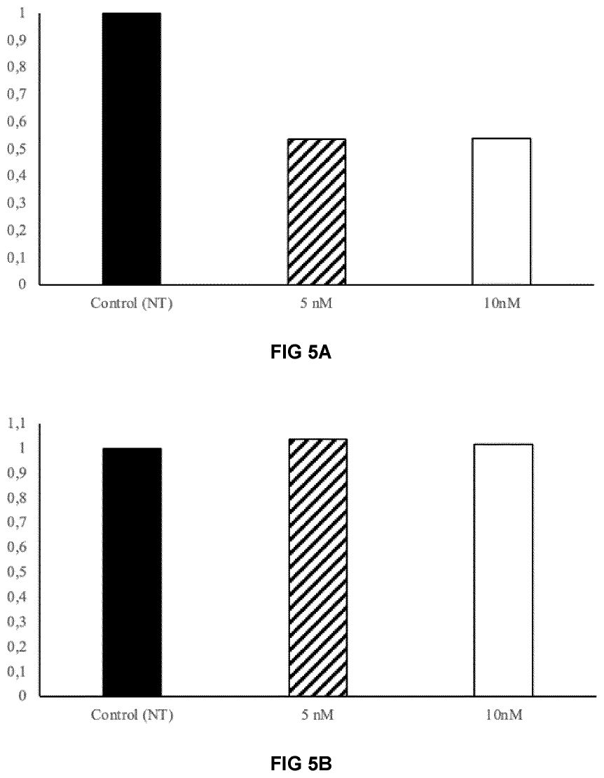

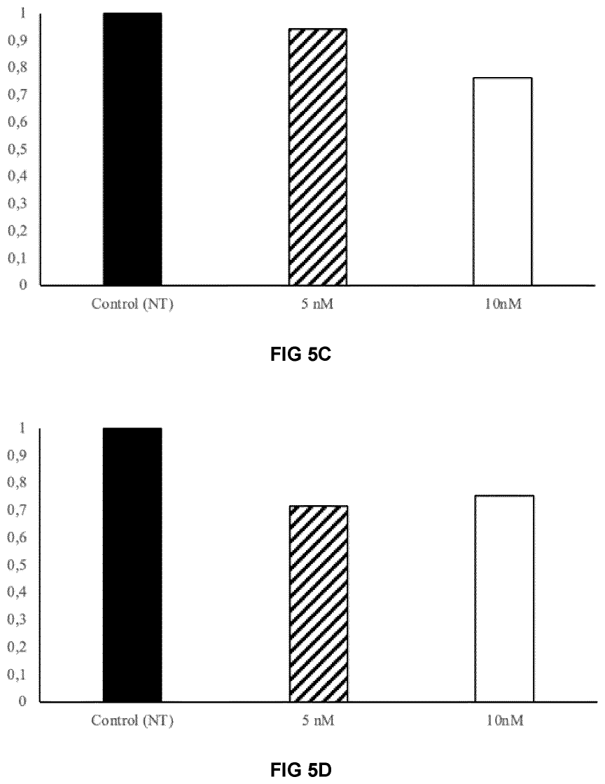

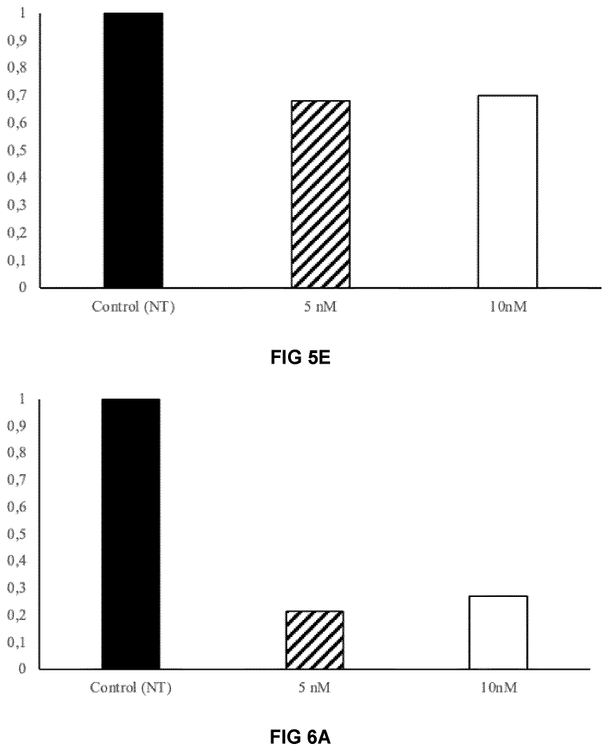

GH levels in the supernatant were quantified using a Sandwich ELISA assay (Millipore Rat/Mouse Growth Hormone ELISA kit #EZRMGH-45K) according to the manufacturer's instructions. This method involved the initial capture of supernatant rat GH to the wells of a microtiter plate coated by a pre-titered amount of anti-Growth Hormone polyclonal antibodies. Unbound GH was washed away and then a second biotinylated anti-GH polyclonal antibody was bound to the captured GH molecules. Unbound GH was again washed away and there followed a step of conjugation of horseradish peroxidase to the immobilized biotinylated antibodies. After a final wash step, immobilized antibody-enzyme conjugates were quantified using horseradish peroxidase activities in the presence of the substrate 3,3′,5,5′-tetramethylbenzidine. The enzyme activity was measured spectrophotometrically. Concentrations of rat GH were derived by interpolation from a reference curve generated in the same assay with reference standards of known concentrations of rat GH. Results are shown in Table 9 and in FIGS. 5A-5E.

TABLE 9
Change in GH Secretion by GH3 Cells Following
Treatment for 72 hours with Compounds 1-5

| | Fold Change in |
|---|---|
| Compound ID | Measured GH* |
| Control (no treatment) | 1.00 |
| Compound 1; 5 nM | 0.29 |
| Compound 1; 10 nM | 0.29 |
| Compound 2; 5 nM | 0.54 |
| Compound 2; 10 nM | 0.54 |
| Compound 3; 5 nM | 1.04 |
| Compound 3; 10 nM | 1.02 |
| Compound 4; 5 nM | 0.94 |
| Compound 4; 10 nM | 0.76 |
| Compound 5; 5 nM | 0.72 |
| Compound 5; 10 nM | 0.75 |

*In comparison to untreated control GH3 cells

### Example 15

Inhibition of Hormone Release from Human Neuroendocrine Tumor (NT-3) Cells

NT-3 is human neuroendocrine cell line that was derived from an advanced pancreatic neuroendocrine tumor that secreted insulin (insulinoma). NT-3 recapitulates many of the key characteristics of human neuroendocrine tumors, such as high differentiation, slow proliferation, and multiple somatostatin receptor expression (Benten et al 2018). Therefore, it was chosen to study the effects of the compounds on relevant measures of neuroendocrine cell function, such as hormone secretion and proliferation.

For the study, 24-well plates were coated with 1 mg/ml of human placenta-derived type IV collagen in 950 μL of PBS) and fully dried. NT3 cells were seeded at a density of 20,000 cells/well in complete culture medium (0.5 ml), including growth factors and incubated overnight at 37° C., with 5% CO₂. After 24 hours, the medium was aspirated, and fresh complete medium (1 ml/well) supplemented with the following drugs were added:
Compound 1;
Compound 2;
Compound 3;
Compound 4;
Compound 5.

All drugs were administered at 5 nM and 10 nM and biological duplicates were studied. The treatment exposure period was 5 days. Untreated cell controls were also studied. After 5 days, the supernatants were collected and deposited into 1.5 ml test tubes that were then centrifuged at 13,400 rpm for 5 minutes to remove any cell residue. The samples were stored at −80° C. until the time of analysis.

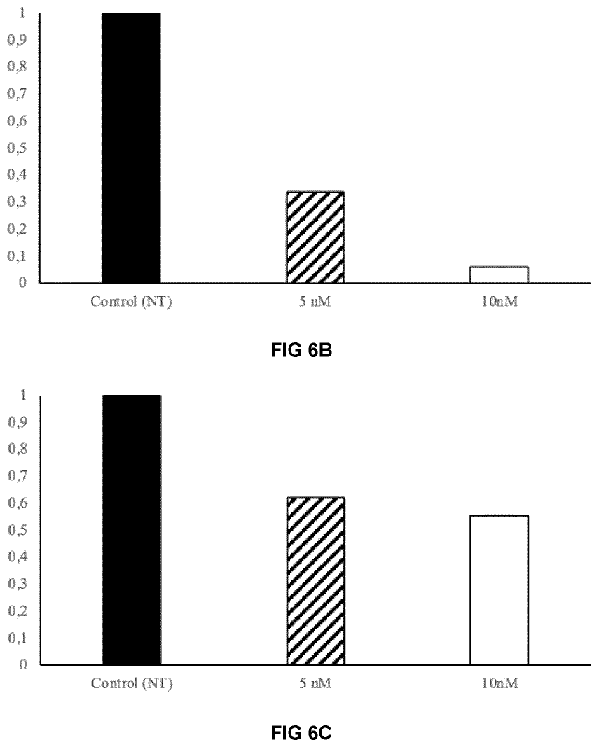

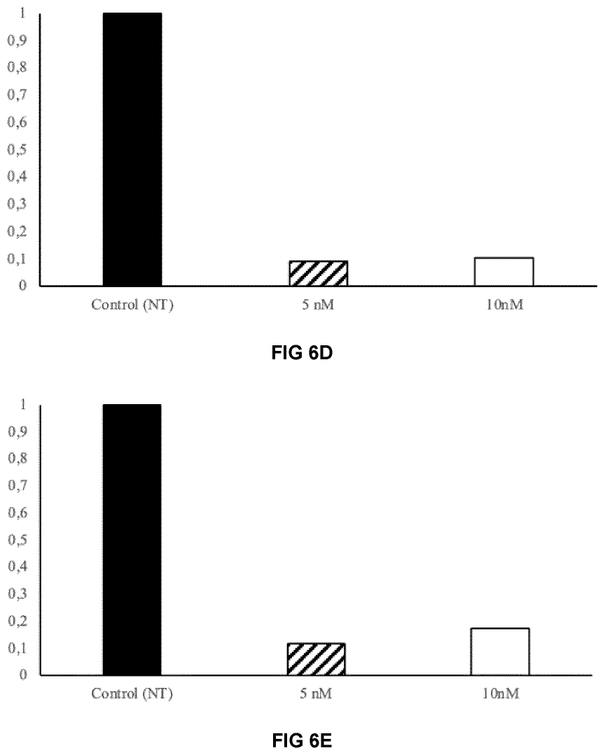

Secreted insulin levels in the supernatant were studied using a Sandwich-ELISA method (Elabscience® Human Insulin ELISA Kit #E-EL-H2665) according to the manufacturer's instructions. A micro-ELISA plate was pre-coated with an antibody specific to human insulin. Samples or Standards were added to the micro-ELISA plate wells and combined with the specific antibody. Then a biotinylated detection antibody specific for human insulin and Avidin-Horseradish Peroxidase (HRP) conjugate were added successively to each micro plate well, incubated and then washed. Substrate solution was added to each well. Only those wells that contain human insulin, biotinylated detection antibody and Avidin-HRP conjugate appear blue in color. The enzyme-substrate reaction was terminated by the addition of stop solution. The optical density (OD) was measured spectrophotometrically at a wavelength of 450 nm±2 nm, and the concentration of human insulin was then derived from the standard curve. Results are shown in Table 10 and in FIGS. 6A-6E

TABLE 10
Change in Insulin Secretion by NT-3 Insulinoma Cells
Following Treatment for 5 Days with Compounds 1-5

| | Fold Change in |
|---|---|
| Compound ID | Measured Insulin* |
| Control (no treatment) | 1.00 |
| Compound 1; 5 nM | 0.22 |
| Compound 1; 10 nM | 0.27 |
| Compound 2; 5 nM | 0.34 |
| Compound 2; 10 nM | 0.06 |
| Compound 3; 5 nM | 0.62 |
| Compound 3; 10 nM | 0.56 |
| Compound 4; 5 nM | 0.09 |
| Compound 4; 10 nM | 0.10 |
| Compound 5; 5 nM | 0.12 |
| Compound 5; 10 nM | 0.18 |

*In comparison to untreated control NT-3 cells

### Example 16

### Inhibition of Proliferation of Human Neuroendocrine Tumor (NT-3) Cells

The water-soluble tetrazolium-1 (WST-1) assay is a standard method for assessing cell viability and proliferation in vitro. The WST-1 assay method is based on the cleavage of tetrazolium salts in culture medium to the dye, formazan, by active mitochondrial enzymes. As viable cell numbers change, so too does the action of mitochondrial dehydrogenases and cleavage of tetrazolium salts. The formation of formazan in the cell medium can be assayed spectrophotometrically as a measure of the number of living cells in the sample well. The effect of various compounds on the cell viability or proliferation rates can be measured as compared with controls or Standards.

The compounds studied were:
Compound 1;
Compound 4;
Compound 5.

All compounds were studied at a dose of 5 nM, and appropriate untreated controls were included.

NT-3 cells were seeded in complete RPMI-1640 medium (100 μl) into a 96-well microplate (20.000 cells/well). This was incubated at 37° C. in 5% CO₂ to allow cells to adhere. Cells were treated with the test compounds at a concentration of 5 nM for a period of 5 days without changing the medium and without adding fresh compounds. A total of 10 μl of the WST-1 reagent was added to each well, in order to achieve a 1:10 ratio and the plate was incubated in the dark at 37° C. for 3 h. The absorbance of the formazan dye was measured at 450 nm and 690 nm using a microplate reader. The cell culture medium was used as a blank control. Average absorbance values were calculated for each group at 450 and 690 nm wavelengths and corrected for blank values. All values were compared to that of the untreated cell group control. Results are shown in Table 11 and in FIG. 7A.

TABLE 11
Change in Proliferation of NT-3 Insulinoma Cells Following
Treatment for 5 Days with Compounds 1, 4 and 5

| | Fold Change in |
|---|---|
| Compound ID | Proliferation* |
| Control (no treatment) | 1.00 |
| Compound 1; 5 nM | 0.55 |
| Compound 4; 5 nM | 0.93 |
| Compound 5; 5 nM | 0.52 |

*In comparison to untreated control NT-3 cells

A number of embodiments of the invention have been described. Nevertheless, it will be understood that various modifications may be made without departing from the spirit and scope of the invention. Accordingly, other embodiments are within the scope of the following claims.

1. A compound of the Formula I, or a pharmaceutically acceptable salt or solvate thereof: 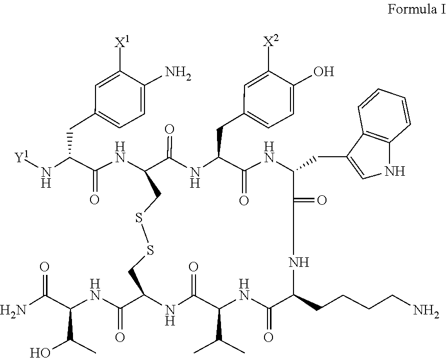
wherein:
X¹ is selected from the group consisting of —F, —Cl, —Br, and —I;
X² is selected from the group consisting of —F, —Cl, —Br, and —I; and
Y¹ is selected from the group consisting of —H, a chelator, L¹-H, -L¹-chelator, an amino protecting group, and a solid support; wherein L¹ is a linker;
provided that X¹ and X² are not both —I.

2. The compound of claim 1, wherein Y¹ is selected from the group consisting of a chelator and -L¹-chelator.

3. The compound of any of claims 1-2, wherein said chelator is complexed to a radionuclide, preferably wherein said radionuclide is selected from the group consisting of boron-10, fluorine-18, phosphorus-32, scandium-47, copper-67, gallium-72, rubidium-82, strontium-89, yttrium-90, technetium-99, palladium-103, indium-111, iodine-125, caesium-131, iodine-131, samarium-153, gadolinium-157, gadolinium-159, terbium-149, samarium-153, terbium-161, dysprosium-165, holmium-166, ytterbium-175, lutetium-177, rhenium-186, rhenium-188, thallium-201, astatine-211, lead-212, bismuth-212, bismuth-213, radium-223, actinium-225, and thorium-227.

4. The compound of claim 1, wherein said compound is of the Formula I-A, or a pharmaceutically acceptable salt or solvate thereof: 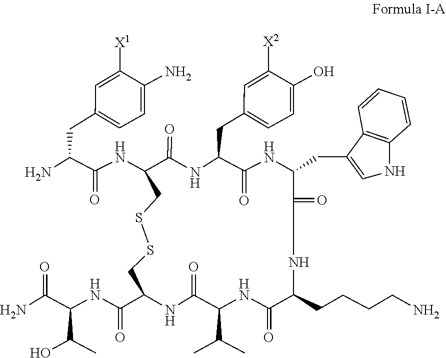
wherein:
X¹ is selected from the group consisting of —F, —Cl, —Br, and —I;
X² is selected from the group consisting of —F, —Cl, —Br, and —I; and
provided that X¹ and X² are not both —I.

5. The compound of any of claims 1-4, wherein X¹ and X² are the same and are both selected from the group consisting of —F, —Cl, and —Br.

6. The compound of any of claims 1-4, wherein said compound is selected from the group consisting of:

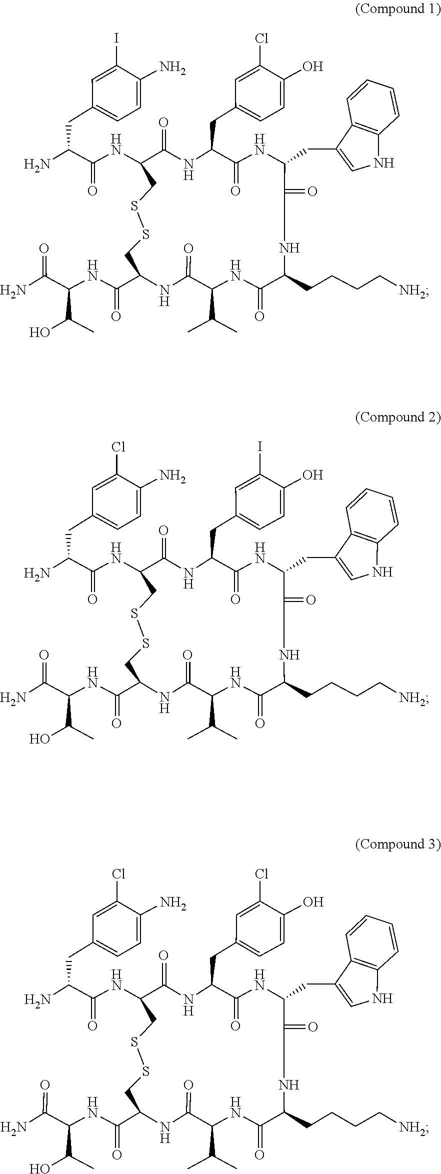

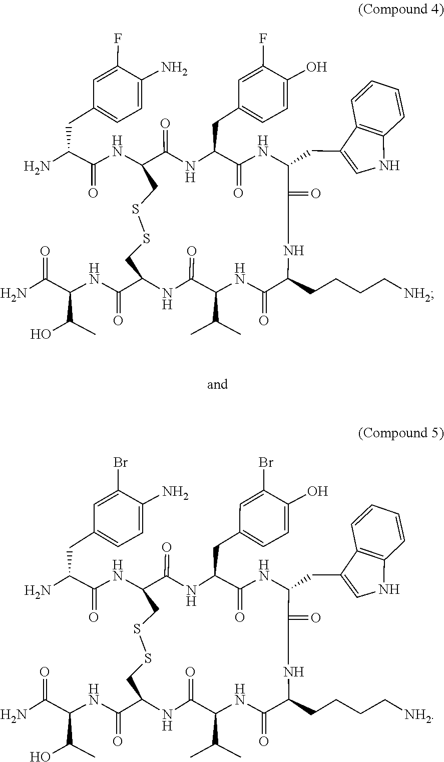 [SMILES: CC(C)[C@@H]1NC(=O)[C@H](CCCCN)NC(=O)[C@@H](Cc2c[nH]c3ccccc23)NC(=O)[C@H](Cc2ccc(O)c(F)c2)NC(=O)[C@H](NC(=O)[C@H](N)Cc2ccc(N)c(F)c2)CSSC[C@H](C(=O)N[C@H](C(N)=O)C(C)O)NC1=O ; CC(C)[C@@H]1NC(=O)[C@H](CCCCN)NC(=O)[C@@H](Cc2c[nH]c3ccccc23)NC(=O)[C@H](Cc2ccc(O)c(Br)c2)NC(=O)[C@H](NC(=O)[C@H](N)Cc2ccc(N)c(Br)c2)CSSC[C@H](C(=O)N[C@H](C(N)=O)C(C)O)NC1=O]

7. The compound of any of claims 1-4, wherein X¹ is —I and X² is —Cl.

8. The compound of any of claims 1-4, wherein X¹ is —Cl and X² is —I.

9. The compound of any of claims 1-4, wherein X¹ is —Cl and X² is —Cl.

10. The compound of any of claims 1-4, wherein X¹ is —F and X² is —F.

11. The compound of any of claims 1-4, wherein X¹ is —Br and X² is —Br.

12. A pharmaceutical composition comprising a compound of any of claims 1 to 11 and a pharmaceutically acceptable carrier.

13. The compound of any of the claims 1 to 11, or the pharmaceutical composition of claim 12, for use as a medicament.

14. A compound of any one of claims 1-11 for use as a medicament.

15. A compound of any one of claims 1-11 for use in a method of treating a SST2 or a SST5 cell proliferative disorder, preferably a proliferative disorder involving cells expressing SST2 and SST5, in a subject.
